(19) United States
(12) Patent Application Publication
(10) Pub. No.: US 2025/0009643 A1
(43) Pub. Date: Jan. 9, 2025

(54) NANOCOMPOSITE COMPOSITIONS AND METHODS OF MAKING AND USING SAME

(71) Applicant: UNIVERSITY OF LOUISIANA OF LAFAYETTE, Lafayette, LA (US)

(72) Inventors: William CHIRDON, Lafayette, LA (US); Nicholas A. BAUDOIN, Lafayette, LA (US)

(73) Assignee: UNIVERSITY OF LOUISIANA OF LAFAYETTE, Lafayette, LA (US)

(21) Appl. No.: 18/610,136

(22) Filed: Mar. 19, 2024

Related U.S. Application Data
(60) Provisional application No. 63/453,728, filed on Mar. 21, 2023.

(51) Int. Cl. A61K 8/99; A61K 8/29; A61Q 5/00; A61Q 19/00

(52) U.S. Cl. CPC A61K 8/99; A61K 8/29; A61Q 5/00; A61Q 19/00

(57) ABSTRACT

Disclosed herein are nanocomposite compositions and methods that include a microalgae or cyanobacterial biomass, where the composition has a content of the microalgae or cyanobacterial biomass from about 0.01 wt % to about 95.00 wt %; nanoparticles, where the nanoparticles include titanium dioxide, and where the composition has a content of the nanoparticles from about 0.01 wt % to about 22.84 wt %; a salt or chelating agent, and where the composition has a content of the nanoparticles from about 0.01 wt % to about 25.01 wt %; an acid, and where the composition has a content of the acid from about 0.01 wt % to about 2.00 wt %; and a carrier medium, and where the composition has a content of the carrier medium from about 47.00 wt % to about 68.00 wt %.

### CROSS-REFERENCE TO RELATED APPLICATIONS

This application claims benefit of priority under 35 U.S.C. § 119(e) of U.S. Ser. No. 63/453,728, filed Mar. 21, 2023, the entire contents of which is incorporated herein by reference in its entirety.

### BACKGROUND

### Field

Nanocomposite compositions and methods for making and using them in personal care products are described.

### Description of the Related Art

With constant innovation in pursuit of more sustainable and healthier products [1,2], the cosmetic industry is among the fastest growing industries [3,4] and is expected to grow in value from $341.1 billion in 2020 to $480.4 billion in 2030 with a capital annual growth rate (CAGR) of 5.1% [5]. Cosmetic products are highly diverse in formulation and applications which include skin care, hair care, body health, makeup, and color cosmetics [6]. Cosmetic emulsions are formulated differently than emulsions in other industries, due to their purpose in delivering pleasant sensorial experiences to consumers as well as providing necessary physicochemical functions: cleansing, nourishment, and hydration. Cosmetic formulations are heavily focused on shelf-life, phase stability, performance of materials, material biodegradability, product appearance, sensory feeling/texture, pH level, temperature sensitivity, and rheological profile [7-9].

Many cosmetic formulations employ surfactants, which can exhibit adverse effects regarding the environment, living cells, and overall health. Surfactants are commonly used in order to formulate and stabilize oil/water emulsions by reducing the interfacial tension between phases and subsequently minimizing the system's free energy [10]. In terms of skin health, recent studies indicated that petroleum-derived surfactants can cause skin irritation, hemolysis, and cytotoxicity [11].

In response to consumer demand and the global initiative in using biodegradable and biocompatible materials, other conventional stabilizers like colloidal particles are gaining in popularity for their application in surfactant-free formulations. Emulsion-based formulations that employ dispersed particles, including nanoparticles, are known as particle-stabilized emulsions or Pickering emulsions [12,13], which have applications in many fields such as cosmetics, pharmaceuticals, food, and agriculture. Particle-stabilized formulations can be advantageous in terms of improving phase stability, skin permeation, sensory effects, control over drug release, rheological profiles, and sustainability [14]. However, surfactant-free formulations can also have drawbacks. For example, surfactant-free formulations can have limited tunability of desired physical properties, which can limit their ability to be used in various commercial products.

Consequently, there is a need for new nanocomposite compositions that can have an expansive tunability of desired physical properties for use in personal care products.

### SUMMARY

Provided herein are nanocomposite compositions and methods that can provide expansive tunability of desired physical properties for commercial products. In a specific embodiment, the nanocomposite composition includes a microalgae or cyanobacterial biomass, where the composition has a content of the microalgae or cyanobacterial biomass from about 50.00 wt % to about 95.00 wt %; nanoparticles, where the nanoparticles include titanium dioxide, and where the composition has a content of the nanoparticles from about 0.01 wt % to about 22.84 wt %; a salt or chelating agent, and where the composition has a content of the nanoparticles from about 0.01 wt % to about 25.01 wt %; an acid, and where the composition has a content of the acid from about 0.01 wt % to about 2.00 wt %; and a carrier medium, and where the composition has a content of the carrier medium from about 47.00 wt % to about 68.00 wt %.

In another specific embodiment, a method of making a nanocomposite composition includes: contacting a microalgae biomass, nanoparticles, salt or chelating agent, an acid, and a carrier material to make the nanocomposite composition, where the composition has a content of the microalgae biomass from about 50.00 wt % to about 95.00 wt %, where the nanoparticles include titanium dioxide, where the composition has a content of the nanoparticles from about 0.01 wt % to about 22.84 wt %, where the composition has a content of the salt or chelating agent is from about 0.01 wt % to about 25.01 wt %, where the composition has a content of the acid from about 0.01 wt % to about 2.00 wt %, and where the composition has a content of the carrier medium from about 47.00 wt % to about 68.00 wt %.

In another specific embodiment, a method of using a nanocomposite composition includes making a nanocomposite composition and applying to a surface.

### BRIEF DESCRIPTION OF THE DRAWINGS

For the purposes of promoting an understanding of the principles of the present disclosure, reference is now made to the embodiments illustrated in the drawings, which are described below. The embodiments disclosed herein are not intended to be exhaustive or limit the present disclosure to the precise form disclosed in the following detailed description. Rather, the embodiments are chosen and described so that others skilled in the art can utilize their teachings. Therefore, no limitation of the scope of the present disclosure is thereby intended.

FIG. 1: Shows Table 1, which lists the nanocomposite compositions initial formulation before heating and final formulations after heating.

FIG. 2: Shows Table 2, which lists the pH, yield stress and average Spread Diameter of nanocomposite compositions.

FIGS. 3A-3H: Show graphs of the amplitude sweep responses for algae-based nanocomposite formulations with varying concentrations of TiO₂, algae, calcium, and citric acid at 20° C. and 1 rad/s. FIG. 3A is a graph of moduli as a function of strain for TiO₂ and FIG. 3B is a graph of moduli as a function of shear stress for TiO₂. FIG. 3C is a graph of a graph of moduli as a function of strain for algae and FIG. 3D is a graph of moduli as a function of shear stress for algae. FIG. 3E is a graph of moduli as a function of strain for calcium and FIG. 3F is a graph of moduli as a function of shear stress for calcium. FIG. 3G is a graph of moduli as a function of strain for citric acid and FIG. 3H is a graph of moduli as a function of shear stress for citric acid.

FIGS. 4A-4H: Show graphs of a frequency sweep responses for algae-based nanocomposite formulations with varying concentrations of TiO₂, algae, calcium, and citric acid at 20° C. and 0.05% strain. FIG. 4A is a graph of moduli as a function of angular frequency for TiO₂ and FIG. 4B is a graph of viscosity as a function of angular frequency with TiO₂. FIG. 4C is a graph of moduli as a function of angular frequency with algae and FIG. 4D is a graph of viscosity as a function of angular frequency with algae. FIG. 4E is a graph of moduli as a function of angular frequency with calcium and FIG. 4F is a graph of viscosity as a function of angular frequency with calcium. FIG. 4G is a graph of moduli as a function of angular frequency with citric acid and FIG. 4H is a graph of viscosity as a function of the angular frequency with citric acid.

FIGS. 5A-5D: Show flow curves of viscosity as a function of shear rate for algae-based nanocomposite formulations with varying concentrations of TiO₂, algae, calcium, and citric acid at 20° C. FIG. 5A is a graph of viscosity as a function of shear rate with TiO₂. FIG. 5B is a graph of viscosity as a function of shear rate with algae. FIG. 5C is a graph of viscosity as a function of shear rate with calcium. FIG. 5D is a graph of viscosity as a function of shear rate with citric acid.

FIGS. 6A-6J: Show graphs of an oscillatory 3-interval-thixotropy test for algae-based nanocomposite formulations with different concentrations of TiO₂, algae, calcium, and citric acid at 20° C. and 70° C. FIGS. 6A and 6B are graphs of moduli as a function of time for titanium dioxide/algae/calcium nanocomposite. FIGS. 6C and 6D are graphs of moduli as a function of time with TiO₂. FIG. 6E and FIG. 6F are graphs of moduli as a function of time with algae. FIG. 6G and FIG. 6H are graphs of moduli as a function of time with calcium. FIGS. 6I and 6J are graphs of time vs moduli for citric acid.

FIG. 7: Shows Table 3, which lists a creep—recovery (20° C.) and thixotropy (20° C. and 70° C.) of nanocomposite compositions.

FIG. 8: Shows a graph of a creep—recovery test for algae-based nanocomposite compositions with different concentrations of TiO₂, algae, calcium, and citric acid at 20° C.

FIG. 9: Shows Table 4, which lists a p-value Data for the Properties of the Algae-based Nanocomposite compositions.

### DETAILED DESCRIPTION

In one or more embodiments, the nanocomposite compositions can include, but are not limited: one or more proteinaceous biomasses, one or more microalgae proteinaceous biomasses, one or more cyanobacterial proteinaceous biomasses, one or more nanoparticles, one or more nanocomposites, one or more titanium dioxides, one or more salts, one or more chelating agents, one or more polymers, one or more acids, one or more bases, one or more surfactants, one or more lubricants, one or more cleansers, one of more astringents, one or more viscosifiers, one or more thinners, one or more dispersants, one or more flocculants, one or more pigment modifiers or coloring agents, one or more pH control additives, one or more pH buffers, one or more emulsifiers, one or more anti-microbial agents, one or more anti-oxidants, one or more flame-retardants, one or more preservative agents, one or more solvents, one or more carrier media, one or more additives, and water. The one or more nanoparticles can include, but are not limited to: one or more cores. The one or more nanocomposites can include, but are not limited to: one or more cores and one or more layers. The nanocomposite compositions can include particle-stabilized formulations that are highly tunable, which can benefit both manufacturing processes and product application.

The one or more proteinaceous biomasses can include, but are not limited to: one or more algae biomass, one or more cyanobacteria biomass, one or more Arthrospira platensis biomass, one or more Spirulina biomass, and mixtures thereof. The one or more microalgae proteinaceous biomasses can include, but are not limited to: whole biomass, biomass after of extraction, such as extractions that increase the protein content of the biomass, biomass with at least a portion of its fat removed, biomass with at least a portion of its carbohydrates removed. In an embodiment, the one or more proteinaceous biomasses can be obtained from a wide variety of sources. For example, the proteinaceous biomasses can be obtained from wastewater streams or as byproduct from commercial processes.

The one or more proteinaceous biomasses can have a protein content that varies widely. For example, the one or more proteinaceous biomasses can have a protein content from a low of about 0.01 wt %, about 0.1 wt %, or about 1.0 wt %, to a high of about 50.0 wt %, about 95.0 wt %, or about 99.9 wt %. In another example, the one or more proteinaceous biomasses can have a protein from about 0.01 wt % to about 99.9 wt %, about 0.01 wt % to about 95.0 wt %, about 0.01 wt % to about 19.0 wt %, about 0.1 wt % to about 2.0 wt %, about 1.0 wt % to about 5.0 wt %, about 2.0 wt % to about 12.0 wt %, about 3.0 wt % to about 20.0 wt %, about 5.0 wt % to about 25.0 wt %, about 10.0 wt % to about 30.0 wt %, about 15.0 wt % to about 40.0 wt %, about 20.0 wt % to about 60.0 wt %, about 25.0 wt % to about 70.0 wt %, about 30.0 wt % to about 80.0 wt %, about 40.0 wt % to about 60.0 wt %, about 45.0 wt % to about 55.0 wt %, about 50.0 wt % to about 95.0 wt %, about 56.0 wt % to about 75.0 wt %, about 65.0 wt % to about 85.0 wt %, about 85 wt % to about 92.0 wt %, about 90.0 wt % to about 99.0 wt %. The content of the proteins in the proteinaceous biomasses can be based on the total weight of the proteinaceous biomasses.

The nanocomposite composition can include a content of the one or more microalgae that can vary widely. For example, the nanocomposite composition can include a content of the microalgae proteinaceous biomass from a low of about 0.01 wt %, about 0.1 wt %, or about 1.0 wt %, to a high of about 50.0 wt %, about 95.0 wt %, or about 99.9 wt %. In another example, the nanocomposite composition can include a content of the microalgae proteinaceous biomass from about 0.01 wt % to about 99.9 wt %, about 0.01 wt % to about 19.0 wt %, about 0.01 wt % to about 95.0 wt %, about 0.1 wt % to about 2.0 wt %, about 1.0 wt % to about 5.0 wt %, about 2.0 wt % to about 12.0 wt %, about 3.0 wt % to about 20.0 wt %, about 5.0 wt % to about 25.0 wt %, about 10.0 wt % to about 30.0 wt %, about 15.0 wt % to about 40.0 wt %, about 20.0 wt % to about 60.0 wt %, about 25.0 wt % to about 70.0 wt %, about 30.0 wt % to about 80.0 wt %, about 40.0 wt % to about 60.0 wt %, about 45.0 wt % to about 55.0 wt %, about 50.0 wt % to about 95.0 wt %, about 56.0 wt % to about 75.0 wt %, about 65.0 wt % to about 85.0 wt %, about 85 wt % to about 92.0 wt %, about 90.0 wt % to about 99.0 wt %. In an embodiment, the nanocomposite composition can be free of the one or more microalgae proteinaceous biomasses. The content of the one or more microalgae proteinaceous biomasses in the nanocomposite composition can be based on the total weight of the nanocomposite composition or based on the one or more microalgae proteinaceous biomasses, one or more cyanobacterial proteinaceous biomasses, one or more nanoparticles, one or more nanocomposites, one or more titanium dioxides, one or more salts, one or more chelating agents, one or more polymers, one or more acids, one or more bases, one or more surfactants, one or more lubricants, one or more cleansers, one of more astringents, one or more viscosifiers, one or more thinners, one or more dispersants, one or more flocculants, one or more pigment modifiers or coloring agents, one or more pH control additives, one or more pH buffers, one or more emulsifiers, one or more anti-microbial agents, one or more anti-oxidants, one or more flame-retardants, one or more preservative agents, one or more solvents, one or more carrier media, one or more additives, and water.

The nanocomposite composition can include a content of the one or more cyanobacterial proteinaceous biomasses that can vary widely. For example, the nanocomposite composition can include a content of the cyanobacterial proteinaceous biomass from a low of about 0.01 wt %, about 0.1 wt %, or about 1.0 wt %, to a high of about 50.0 wt %, about 95.0 wt %, or about 99.9 wt %. In another example, the nanocomposite composition can include a content of the cyanobacterial proteinaceous biomass from about 0.01 wt % to about 99.9 wt %, about 0.01 wt % to about 19.0 wt %, about 0.01 wt % to about 95.0 wt %, about 0.1 wt % to about 2.0 wt %, about 1.0 wt % to about 5.0 wt %, about 2.0 wt % to about 12.0 wt %, about 3.0 wt % to about 20.0 wt %, about 5.0 wt % to about 25.0 wt %, about 10.0 wt % to about 30.0 wt %, about 15.0 wt % to about 40.0 wt %, about 20.0 wt % to about 60.0 wt %, about 25.0 wt % to about 70.0 wt %, about 30.0 wt % to about 80.0 wt %, about 40.0 wt % to about 60.0 wt %, about 45.0 wt % to about 55.0 wt %, about 50.0 wt % to about 95.0 wt %, about 56.0 wt % to about 75.0 wt %, about 65.0 wt % to about 85.0 wt %, about 85 wt % to about 92.0 wt %, about 90.0 wt % to about 99.0 wt %. In an embodiment, the nanocomposite composition can be free of the one or more cyanobacterial proteinaceous biomass. The content of the one or more cyanobacterial proteinaceous biomass in the nanocomposite composition can be based on the total weight of the nanocomposite composition or based on the total weight of the nanocomposite composition or based on the one or more microalgae proteinaceous biomasses, one or more cyanobacterial proteinaceous biomasses, one or more nanoparticles, one or more nanocomposites, one or more titanium dioxides, one or more salts, one or more chelating agents, one or more polymers, one or more acids, one or more bases, one or more surfactants, one or more lubricants, one or more cleansers, one of more astringents, one or more viscosifiers, one or more thinners, one or more dispersants, one or more flocculants, one or more pigment modifiers or coloring agents, one or more pH control additives, one or more pH buffers, one or more emulsifiers, one or more anti-microbial agents, one or more anti-oxidants, one or more flame-retardants, one or more preservative agents, one or more solvents, one or more carrier media, one or more additives, and water.

The nanocomposite composition can include a content of the one or more nanoparticles that can vary widely. For example, the nanocomposite composition can include a content of the nanoparticles from a low of about 0.01 wt %, about 0.1 wt %, or about 1.0 wt %, to a high of about 20.0 wt %, about 50.0 wt %, or about 99.9 wt %. In another example, the nanocomposite composition can include a content of the nanoparticles from about 0.01 wt % to about 99.9 wt %, about 0.01 wt % to about 23.0 wt %, about 0.1 wt % to about 2.0 wt %, about 1.0 wt % to about 5.0 wt %, about 2.0 wt % to about 12.0 wt %, about 3.0 wt % to about 20.0 wt %, about 5.0 wt % to about 25.0 wt %, about 10.0 wt % to about 30.0 wt %, about 15.0 wt % to about 40.0 wt %, about 20.0 wt % to about 60.0 wt %, about 25.0 wt % to about 70.0 wt %, about 30.0 wt % to about 80.0 wt %, about 40 wt % to about 60.0 wt %, about 45 wt % to about 55.0 wt %, about 56 wt % to about 75.0 wt %, about 65 wt % to about 85.0 wt %, about 85 wt % to about 92.0 wt %, about 90 wt % to about 99.0 wt %. In an embodiment, the nanocomposite composition can be free of the one or more nanoparticles. The weight percent of the one or more nanoparticles in the nanocomposite composition can be based on the total weight of the nanocomposite composition or based on the total weight of the nanocomposite composition or based on the one or more proteinaceous biomasses, one or more microalgae proteinaceous biomasses, one or more cyanobacterial proteinaceous biomasses, one or more nanoparticles, one or more nanocomposites, one or more titanium dioxides, one or more salts, one or more chelating agents, one or more polymers, one or more acids, one or more bases, one or more surfactants, one or more lubricants, one or more cleansers, one of more astringents, one or more viscosifiers, one or more thinners, one or more dispersants, one or more flocculants, one or more pigment modifiers or coloring agents, one or more pH control additives, one or more pH buffers, one or more emulsifiers, one or more anti-microbial agents, one or more anti-oxidants, one or more flame-retardants, one or more preservative agents, one or more solvents, one or more carrier media, one or more additives, and water.

The one or more nanoparticles can have an average particle size that varies widely. For example, the one or more nanoparticles can have an average diameter from a low of about 1 nanometers (nm), about 5 nm, or about 25 nm, to a high of about 100 nm, about 200 nm, or about 500 nm. In another example, the nanoparticles can have an average diameter from about 1 nm to about 500 nm, about 1 nm to about 5 nm, about 5 nm to about 500 nm, about 15 nm to about 50 nm, about 20 nm to about 50 nm, about 25 nm to about 60 nm, about 30 nm to about 200 nm, about 40 nm to about 250 nm, about 70 nm to about 350 nm, about 100 nm to about 400 nm, about 100 nm to about 250 nm, or about 132 nm to about 500 nm.

The one or more titanium dioxides can include, but are not limited to: one or more nanoparticles. The nanocomposite composition can include a content of the one or more titanium dioxides that can vary widely. For example, the nanocomposite composition can include a content of the titanium dioxide from a low of about 0.01 wt %, about 0.1 wt %, or about 1.0 wt %, to a high of about 20.0 wt %, about 50.0 wt %, or about 99.9 wt %. In another example, the nanocomposite composition can include a content of the titanium dioxide from about 0.01 wt % to about 99.9 wt %, about 0.01 wt % to about 23.0 wt %, about 0.1 wt % to about 2.0 wt %, about 1.0 wt % to about 5.0 wt %, about 2.0 wt % to about 12.0 wt %, about 3.0 wt % to about 20.0 wt %, about 5.0 wt % to about 25.0 wt %, about 10.0 wt % to about 30.0 wt %, about 15.0 wt % to about 40.0 wt %, about 20.0 wt % to about 60.0 wt %, about 25.0 wt % to about 70.0 wt %, about 30.0 wt % to about 80.0 wt %, about 40 wt % to about 60.0 wt %, about 45 wt % to about 55.0 wt %, about 56 wt % to about 75.0 wt %, about 65 wt % to about 85.0 wt %, about 85 wt % to about 92.0 wt %, about 90 wt % to about 99.0 wt %. In an embodiment, the nanocomposite composition can be free of the one or more titanium dioxides. The weight percent of the one or more titanium dioxides in the nanocomposite composition can be based on the total weight of the nanocomposite composition or based on the total weight of the nanocomposite composition or based on the one or more proteinaceous biomasses, one or more microalgae proteinaceous biomasses, one or more cyanobacterial proteinaceous biomasses, one or more nanoparticles, one or more nanocomposites, one or more titanium dioxides, one or more salts, one or more chelating agents, one or more polymers, one or more acids, one or more bases, one or more surfactants, one or more lubricants, one or more cleansers, one of more astringents, one or more viscosifiers, one or more thinners, one or more dispersants, one or more flocculants, one or more pigment modifiers or coloring agents, one or more pH control additives, one or more pH buffers, one or more emulsifiers, one or more anti-microbial agents, one or more anti-oxidants, one or more flame-retardants, one or more preservative agents, one or more solvents, one or more carrier media, one or more additives, and water.

The one or more salts can include, but are not limited to: cesium formate (HCOOCs), sodium chloride (NaCl), sodium carbonate (Na₂CO₃), sodium bicarbonate (NaHCO₃), potassium chloride (KCl), potassium carbonate (K₂CO₃), potassium bicarbonate (KHCO₃), potassium fluoride (KF), sodium fluoride (NaF), potassium formate (HCOOK), sodium formate (HCOONa), calcium chloride (CaCl₂), ammonium carbonate ((NH₄)₂CO₃), ammonium chloride (NH₄Cl), tetramethylammonium chloride (N(CH₃)₄Cl), sodium chloride (NaCl), potassium chloride (KCl), dipotassium glutarate (C₅H₆K₂O₄), disodium glutarate (C₅H₆K₂O₄), sodium citrate (Na₃C₆H₅O₇), potassium citrate (K₃C₆H₅O₇), potassium acetate (CH₃CO₂K), choline chloride [((CH₃)₃NCH₂CH₂OH)Cl], sodium acetate (CH₃CO₂Na), sodium sulfate (Na₂SO₄), magnesium chloride (MgCl₂), potassium cocoate, magnesium citrate, magnesium lactate, acid salts, and mixtures thereof.

The nanocomposite composition can include a content of the one or more salts that can vary widely. For example, the nanocomposite composition can include a content of the salt from a low of about 0.01 wt %, about 0.1 wt %, or about 1.0 wt %, to a high of about 20.0 wt %, about 50.0 wt %, or about 99.9 wt %. In another example, the nanocomposite composition can include a content of the salt from about 0.01 wt % to about 99.9 wt %, about 0.01 wt % to about 10.0 wt %, about 0.1 wt % to about 2.0 wt %, about 1.0 wt % to about 5.0 wt %, about 2.0 wt % to about 12.0 wt %, about 3.0 wt % to about 20.0 wt %, about 5.0 wt % to about 25.0 wt %, about 10.0 wt % to about 30.0 wt %, about 15.0 wt % to about 40.0 wt %, about 20.0 wt % to about 60.0 wt %, about 25.0 wt % to about 70.0 wt %, about 30.0 wt % to about 80.0 wt %, about 40 wt % to about 60.0 wt %, about 45 wt % to about 55.0 wt %, about 56 wt % to about 75.0 wt %, about 65 wt % to about 85.0 wt %, about 85 wt % to about 92.0 wt %, about 90 wt % to about 99.0 wt %. In an embodiment, the nanocomposite composition can be free of the one or more salts. The weight percent of the one or more salts in the nanocomposite composition can be based on the total weight of the nanocomposite composition or based on the total weight of the nanocomposite composition or based on the one or more proteinaceous biomasses, one or more microalgae proteinaceous biomasses, one or more cyanobacterial proteinaceous biomasses, one or more nanoparticles, one or more nanocomposites, one or more titanium dioxides, one or more salts, one or more chelating agents, one or more polymers, one or more acids, one or more bases, one or more surfactants, one or more lubricants, one or more cleansers, one of more astringents, one or more viscosifiers, one or more thinners, one or more dispersants, one or more flocculants, one or more pigment modifiers or coloring agents, one or more pH control additives, one or more pH buffers, one or more emulsifiers, one or more anti-microbial agents, one or more anti-oxidants, one or more flame-retardants, one or more preservative agents, one or more solvents, one or more carrier media, one or more additives, and water.

The one or more acids can include, but are not limited to: one or more alpha hydroxy acids (AHAs), hydrochloric acid (HCl), phosphoric acid (H₃PO₄), sulfuric acid (H₂SO₄), carbonic acid (H₂CO₃), formic acid (CH₂O₂), citric acid (C₆H₈O₇), beta hydroxy acids (BHAs), lactic acid (C₃H₆O₃), glycolic acid (C₂H₄O₃)hyaluronic acid (C₁₄H₂₁NO₁₁)nn, salicylic acid (C₇H₆O₃), mandelic acid (C₈H₈O₃), azelaic acid (C₉H₁₆O₄), kojic acid (C₆H₆O₄), ferulic acid (C₁₀H₁₀O₄), trethocanic acid (C₁₅H₃₀O₃), malic acid (C₄H₆O₅), ascorbic acid (C₆H₈O₆), oxalic acid (H₂C₂O₄), benzoic acid (C₇H₆O₂), and mixtures thereof.

The nanocomposite composition can include a content of the one or more acids that can vary widely. For example, the nanocomposite composition can include a content of the acid from a low of about 0.01 wt %, about 0.1 wt %, or about 1.0 wt %, to a high of about 20.0 wt %, about 50.0 wt %, or about 99.9 wt %. In another example, the nanocomposite composition can include a content of the acid from about 0.01 wt % to about 99.9 wt %, about 0.01 wt % to about 2.0 wt %, about 0.1 wt % to about 2.0 wt %, about 1.0 wt % to about 5.0 wt %, about 2.0 wt % to about 12.0 wt %, about 3.0 wt % to about 20.0 wt %, about 5.0 wt % to about 25.0 wt %, about 10.0 wt % to about 30.0 wt %, about 15.0 wt % to about 40.0 wt %, about 20.0 wt % to about 60.0 wt %, about 25.0 wt % to about 70.0 wt %, about 30.0 wt % to about 80.0 wt %, about 40 wt % to about 60.0 wt %, about 45 wt % to about 55.0 wt %, about 56 wt % to about 75.0 wt %, about 65 wt % to about 85.0 wt %, about 85 wt % to about 92.0 wt %, about 90 wt % to about 99.0 wt %. In an embodiment, the nanocomposite composition can be free of the one or more acids. The weight percent of the one or more acids in the nanocomposite composition can be based on the total weight of the nanocomposite composition or based on the total weight of the nanocomposite composition or based on the one or more proteinaceous biomasses, one or more microalgae proteinaceous biomasses, one or more cyanobacterial proteinaceous biomasses, one or more nanoparticles, one or more nanocomposites, one or more titanium dioxides, one or more salts, one or more chelating agents, one or more polymers, one or more acids, one or more bases, one or more surfactants, one or more lubricants, one or more cleansers, one of more astringents, one or more viscosifiers, one or more thinners, one or more dispersants, one or more flocculants, one or more pigment modifiers or coloring agents, one or more pH control additives, one or more pH buffers, one or more emulsifiers, one or more anti-microbial agents, one or more anti-oxidants, one or more flame-retardants, one or more preservative agents, one or more solvents, one or more carrier media, one or more additives, and water.

The one or more bases can include, but are not limited to: calcium hydroxide [Ca(OH)₂], sodium hydroxide (NaOH), potassium hydroxide (KOH), and mixtures thereof.

The nanocomposite composition can include a content of the one or more bases that can vary widely. For example, the nanocomposite composition can include a content of the base from a low of about 0.01 wt %, about 0.1 wt %, or about 1.0 wt %, to a high of about 20.0 wt %, about 50.0 wt %, or about 99.9 wt %. In another example, the nanocomposite composition can include a content of the base from about 0.01 wt % to about 99.9 wt %, about 0.01 wt % to about 2.0 wt %, about 0.1 wt % to about 2.0 wt %, about 1.0 wt % to about 5.0 wt %, about 2.0 wt % to about 12.0 wt %, about 3.0 wt % to about 20.0 wt %, about 5.0 wt % to about 25.0 wt %, about 10.0 wt % to about 30.0 wt %, about 15.0 wt % to about 40.0 wt %, about 20.0 wt % to about 60.0 wt %, about 25.0 wt % to about 70.0 wt %, about 30.0 wt % to about 80.0 wt %, about 40 wt % to about 60.0 wt %, about 45 wt % to about 55.0 wt %, about 56 wt % to about 75.0 wt %, about 65 wt % to about 85.0 wt %, about 85 wt % to about 92.0 wt %, about 90 wt % to about 99.0 wt %. In an embodiment, the nanocomposite composition can be free of the one or more bases. The weight percent of the one or more bases in the nanocomposite composition can be based on the total weight of the nanocomposite composition or based on the total weight of the nanocomposite composition or based on the one or more proteinaceous biomasses, one or more microalgae proteinaceous biomasses, one or more cyanobacterial proteinaceous biomasses, one or more nanoparticles, one or more nanocomposites, one or more titanium dioxides, one or more salts, one or more chelating agents, one or more polymers, one or more acids, one or more bases, one or more surfactants, one or more lubricants, one or more cleansers, one of more astringents, one or more viscosifiers, one or more thinners, one or more dispersants, one or more flocculants, one or more pigment modifiers or coloring agents, one or more pH control additives, one or more pH buffers, one or more emulsifiers, one or more anti-microbial agents, one or more anti-oxidants, one or more flame-retardants, one or more preservative agents, one or more solvents, one or more carrier media, one or more additives, and water.

The one or more solvents and/or carrier media can include, but are not limited to: water, hexanes, toluene, methanol, ethanol, propanol, isopropanol, acetone, acetonitrile, chloroform, diethyl ether, methylene chloride, dimethyl formamide, ethylene glycol, propylene glycol, triethylamine, tetrahydrofuran, and mixtures thereof.

The nanocomposite composition can include a content of the one or more solvents and/or carrier media that can vary widely. For example, the nanocomposite composition can include a content of the solvent and/or carrier media from a low of about 0.01 wt %, about 0.1 wt %, or about 1.0 wt %, to a high of about 20.0 wt %, about 50.0 wt %, or about 99.9 wt %. In another example, the nanocomposite composition can include a content of the solvent and/or carrier media from about 0.01 wt % to about 99.9 wt %, about 0.1 wt % to about 2.0 wt %, about 1.0 wt % to about 5.0 wt %, about 2.0 wt % to about 12.0 wt %, about 3.0 wt % to about 20.0 wt %, about 5.0 wt % to about 25.0 wt %, about 10.0 wt % to about 30.0 wt %, about 15.0 wt % to about 40.0 wt %, about 20.0 wt % to about 60.0 wt %, about 25.0 wt % to about 70.0 wt %, about 30.0 wt % to about 80.0 wt %, about 40 wt % to about 60.0 wt %, about 47.91 wt % to about 67.17 wt %, about 48.0 wt % to about 68.0 wt %, about 45 wt % to about 55.0 wt %, about 56 wt % to about 75.0 wt %, about 65 wt % to about 85.0 wt %, about 85 wt % to about 92.0 wt %, about 90 wt % to about 99.0 wt %. In an embodiment, the nanocomposite composition can be free of the one or more solvents and/or carrier media. The weight percent of the one or more solvents in the nanocomposite composition can be based on the total weight of the nanocomposite composition or based on the one or more proteinaceous biomasses, one or more microalgae proteinaceous biomasses, one or more cyanobacterial proteinaceous biomasses, one or more nanoparticles, one or more nanocomposites, one or more titanium dioxides, one or more salts, one or more chelating agents, one or more polymers, one or more acids, one or more bases, one or more surfactants, one or more lubricants, one or more cleansers, one of more astringents, one or more viscosifiers, one or more thinners, one or more dispersants, one or more flocculants, one or more pigment modifiers or coloring agents, one or more pH control additives, one or more pH buffers, one or more emulsifiers, one or more anti-microbial agents, one or more anti-oxidants, one or more flame-retardants, one or more preservative agents, one or more solvents, one or more carrier media, one or more additives, and water.

The one or more polymers can include, but are not limited to: one or more polypropylenes, one or more polystyrenes, one or more nylons, one or more polyurethanes, one or more polypropylene glycols, one or more polyacrylates, one or more polyacrylamides, one or more starches, one or more polysaccharides, one or more poly alpha olefins, one or more xanthan gums, one or more guar gum, one or more carrageenan, one or more polyvinyls, one or more alginates, one or more pectines, one or more hydroxyethylcelluloses, one or more methyl celluloses, one or more gelatins, and mixtures thereof.

The one or more polymers can have a weight-average molecular weight (M_{W}) that varies widely. For example, the polymers can have a weight-average molecular weight from a low of about 10,000 g/mol, about 35,000 g/mol, or about 40,000 g/mol, to a high of about 800,000 g/mol, about 900,000 g/mol, or about 1,200,000 g/mol. In another example, the polymers can have a weight-average molecular weight that is less than 80,000 g/mol, less than 60,000 g/mol, or less than 50,000 g/mol. In another example, the polymers can have a weight-average molecular weight from about 8,000 g/mol to about 250,000 g/mol, about 30,000 g/mol to about 1,200,000 g/mol, about 20,000 g/mol to about 80,000 g/mol, about 40,000 g/mol to about 80,000 g/mol, about 100,000 g/mol to about 750,000 g/mol, about 480,000 g/mol to about 1,100,000 g/mol, about 500,000 g/mol to about 1,000,000 g/mol. The molecular weight of the polymers can be measured using gel permeation chromatography.

The nanocomposite composition can include a content of the one or more polymers that can vary widely. For example, the nanocomposite composition can include a content of the polymer from a low of about 0.01 wt %, about 0.1 wt %, or about 1.0 wt %, to a high of about 20.0 wt %, about 50.0 wt %, or about 99.9 wt %. In another example, the nanocomposite composition can include a content of the polymer from about 0.01 wt % to about 99.9 wt %, about 0.1 wt % to about 2.0 wt %, about 1.0 wt % to about 5.0 wt %, about 2.0 wt % to about 12.0 wt %, about 3.0 wt % to about 20.0 wt %, about 5.0 wt % to about 25.0 wt %, about 10.0 wt % to about 30.0 wt %, about 15.0 wt % to about 40.0 wt %, about 20.0 wt % to about 60.0 wt %, about 25.0 wt % to about 70.0 wt %, about 30.0 wt % to about 80.0 wt %, about 40 wt % to about 60.0 wt %, about 45 wt % to about 55.0 wt %, about 56 wt % to about 75.0 wt %, about 65 wt % to about 85.0 wt %, about 85 wt % to about 92.0 wt %, about 90 wt % to about 99.0 wt %. In an embodiment, the nanocomposite composition can be free of the one or more polymers. The weight percent of the one or more polymers in the nanocomposite composition can be based on the total weight of the nanocomposite composition based on the total weight of the nanocomposite composition or based on the one or more proteinaceous biomasses, one or more microalgae proteinaceous biomasses, one or more cyanobacterial proteinaceous biomasses, one or more nanoparticles, one or more nanocomposites, one or more titanium dioxides, one or more salts, one or more chelating agents, one or more polymers, one or more acids, one or more bases, one or more surfactants, one or more lubricants, one or more cleansers, one of more astringents, one or more viscosifiers, one or more thinners, one or more dispersants, one or more flocculants, one or more pigment modifiers or coloring agents, one or more pH control additives, one or more pH buffers, one or more emulsifiers, one or more anti-microbial agents, one or more anti-oxidants, one or more flame-retardants, one or more preservative agents, one or more solvents, one or more carrier media, one or more additives, and water.

The one or more additives can include, but are not limited to: one or more coloring agents, one or more viscosifiers, one or more thinners, one or more dispersants, one or more flocculants, one or more pH control additives, one or more lubricants, one or more emulsifiers, one or more surfactants, one or more cleansers, one of more astringents, one or more pigment modifiers or coloring agents, one or more pH buffers, one or more anti-microbial agents, one or more anti-oxidants, one or more flame-retardants, one or more preservative agents, and mixtures thereof.

The nanocomposite composition can include a content of the one or more additives that can vary widely. For example, the nanocomposite composition can include a content of additives from a low of about 0.01 wt %, about 0.1 wt %, or about 1.0 wt %, to a high of about 20.0 wt %, about 50.0 wt %, or about 99.9 wt %. In another example, the nanocomposite composition can include a content of the additive from about 0.01 wt % to about 99.9 wt %, about 0.1 wt % to about 2.0 wt %, about 1.0 wt % to about 5.0 wt %, about 2.0 wt % to about 12.0 wt %, about 3.0 wt % to about 20.0 wt %, about 5.0 wt % to about 25.0 wt %, about 10.0 wt % to about 30.0 wt %, about 15.0 wt % to about 40.0 wt %, about 20.0 wt % to about 60.0 wt %, about 25.0 wt % to about 70.0 wt %, about 30.0 wt % to about 80.0 wt %, about 40 wt % to about 60.0 wt %, about 45 wt % to about 55.0 wt %, about 56 wt % to about 75.0 wt %, about 65 wt % to about 85.0 wt %, about 85 wt % to about 92.0 wt %, about 90 wt % to about 99.0 wt %. In an embodiment, the nanocomposite composition can be free of the one or more additives. The weight percent of the one or more additives in the nanocomposite composition can be based on the total weight of the nanocomposite composition or based on the one or more proteinaceous biomasses, one or more microalgae proteinaceous biomasses, one or more cyanobacterial proteinaceous biomasses, one or more nanoparticles, one or more nanocomposites, one or more titanium dioxides, one or more salts, one or more chelating agents, one or more polymers, one or more acids, one or more bases, one or more surfactants, one or more lubricants, one or more cleansers, one of more astringents, one or more viscosifiers, one or more thinners, one or more dispersants, one or more flocculants, one or more pigment modifiers or coloring agents, one or more pH control additives, one or more pH buffers, one or more emulsifiers, one or more anti-microbial agents, one or more anti-oxidants, one or more flame-retardants, one or more preservative agents, one or more solvents, one or more carrier media, one or more additives, and water.

The nanocomposite composition can include a content of water that can vary widely. For example, the nanocomposite composition can include a content of water from a low of about 0.01 wt %, about 0.1 wt %, or about 1.0 wt %, to a high of about 20.0 wt %, about 50.0 wt %, or about 99.9 wt %. In another example, the nanocomposite composition can include a content of water from about 0.01 wt % to about 99.9 wt %, about 0.1 wt % to about 2.0 wt %, about 1.0 wt % to about 5.0 wt %, about 2.0 wt % to about 12.0 wt %, about 3.0 wt % to about 20.0 wt %, about 5.0 wt % to about 25.0 wt %, about 10.0 wt % to about 30.0 wt %, about 15.0 wt % to about 40.0 wt %, about 20.0 wt % to about 60.0 wt %, about 25.0 wt % to about 70.0 wt %, about 30.0 wt % to about 80.0 wt %, about 40 wt % to about 60.0 wt %, about 47.91 wt % to about 67.17 wt %, about 48.0 wt % to about 68.0 wt %, about 45 wt % to about 55.0 wt %, about 56 wt % to about 75.0 wt %, about 65 wt % to about 85.0 wt %, about 85 wt % to about 92.0 wt %, about 90 wt % to about 99.0 wt %. In an embodiment, the nanocomposite composition can be free of water. The weight percent of the water in the nanocomposite composition can be based on the total weight of the nanocomposite composition or based on the one or more proteinaceous biomasses, one or more microalgae proteinaceous biomasses, one or more cyanobacterial proteinaceous biomasses, one or more nanoparticles, one or more nanocomposites, one or more titanium dioxides, one or more salts, one or more chelating agents, one or more polymers, one or more acids, one or more bases, one or more surfactants, one or more lubricants, one or more cleansers, one of more astringents, one or more viscosifiers, one or more thinners, one or more dispersants, one or more flocculants, one or more pigment modifiers or coloring agents, one or more pH control additives, one or more pH buffers, one or more emulsifiers, one or more anti-microbial agents, one or more anti-oxidants, one or more flame-retardants, one or more preservative agents, one or more solvents, one or more carrier media, one or more additives, and water.

The one or more nanocomposite compositions can have a pH that can vary widely. For example, the nanocomposite composition can have a pH from a low of about 0.00, about 1.00, or about 2.00, to a high of about 12.00, about 13.00, or about 14.00. In another example, the nanocomposite composition can have a pH from about 1.40 to about 7.00, about 0.01 to about 14.00, about 1.00 to about 4.00, about 2.00 to about 7.00, about 2.50 to about 7.50, about 3.00 to about 8.00, about 4.50 to about 11.50, about 5.00 to about 10.00, about 7.50 to about 10.50, about 7.50 to about 10.50, about 8.50 to about 12.50, or about 9.00 to about 13.50.

The one or more nanocomposite compositions can have a yield stress that can vary widely. For example, the nanocomposite composition can have a yield stress from a low of about 0.10 Pa, about 1.00 Pa, or about 10.00 Pa, to a high of about 2,500.00 Pa, about 5,500.00 Pa, or about 12,500.00 Pa. In another example, the nanocomposite composition can have a yield stress from a low of about 0.18 Pa to about 2,380.00 Pa, about 0.55 Pa to about 12,500.00 Pa, about 128.00 Pa to about 4,080.00 Pa, about 80.00 Pa to about 6,380.00 Pa, about 500.00 Pa to about 1,00.00 Pa, about 100.00 Pa to about 6,200.00 Pa, or about 750.00 Pa to about 7,980.00 Pa. In another example, the nanocomposite composition can have a yield stress of greater than 0.18 Pa.

The one or more nanocomposite compositions can have a viscosity that can vary widely. For example, the nanocomposite composition can have a viscosity from a low of about 0.1 cP, about 1.0 cP, or about 10.0 cP, to a high of about 12,000.0 cP, about 500,000.0 cP, or about 2,500,000.0 cP. In another example, the nanocomposite composition can have a viscosity from about 0.1 cP to about 2,500,000.0 cP, about 0.1 cP to about 12,500.0 cP, about 10.0 cP to about 10,000 cP, about 1,000.0 cP to about 250,000.0 cP, about 2,500.0 cP to about 250,000.0 cP, about 2,500.0 cP to about 200,000.0 cP, about 10,000.0 cP to about 100,000.0 cP, about 10,000.0 cP to about 50,000.0 cP, about 100,000.0 cP to about 250,000.0 cP, about 620,000.0 cP to about 850,000.0 cP, about 700,000.0 cP to about 750,000.0 cP, about 700,000.0 cP to about 800,000.0 cP, about 650,000.0 cP to about 855,000.0 cP, about 700,000.0 cP to about 800,000.0 cP, about 500,000.0 cP to about 1,000,000.0 cP, or about 500,000.0 cP to about 2,500,000.0 cP. In another example, the nanocomposite composition can have a viscosity measured over a shear rate from about 0.01 to about 10,000 l/s. The viscosity of the clay swelling inhibitor composition can be measured on a Brookfield viscosimeter. The viscosity of the clay swelling inhibitor compositions can be measured at various temperatures, such as 25° C., 40° C., 60° C., and 100° C.

The one or more nanocomposite compositions can have physical properties that vary widely. For example, the one or more nanocomposite compositions can have: a yield stress of greater than about 0.18 Pa or about 0.18 Pa to about 2,380 Pa; a moduli values from about 0.1 to about 177,300 Pa; a moduli recoveries from about 0.1% to about 100%; a heat-induced moduli magnitude increase of greater than or equal to about 2,693%; a zero-shear viscosities from about 0.1 Pa·s to about 10,000,000 Pa·s; a moduli recoveries from about 0.1% to about 49.58%; a LVER (@ 1 rad/s) from about 0.1 [strain %] to about 10 [strain %]; a crossover point from about 5.51 [strain %] to about 99.1 [strain %]; an amplitude G′ (@ 1 rad/s) of greater than or equal to about 319,000 Pa; an amplitude G″ (@ 1 rad/s) of greater than or equal to about 124,000 Pa; a frequency G′ (0.1-100 rad/s) of greater than or equal to about 405,000 Pa; a frequency G″ (0.1-100 rad/s) of greater than or equal to about 147,000 Pa; a complex viscosity at about 0.1 rad/s to about 100 rad/s from about 0.487 Pa·s to about 3,100,000 Pa·s; a shear stress at about 0.1 l/s to about 10,000 l/s from about 4.98 Pa to about 19,470 Pa; a viscosity evaluated at shear rates from 0.1 l/s to about 10,000 l/s having values from about 0.00561 Pa·s to about 1,770 Pa·s; a consistency index “k” from about 0.59 Pa·s to about 1,308 Pa·s; a flow index “n” from about 0.095 to about 0.496; an average glass-plate spread diameter “d” from about 2.00 cm to about 10.67 cm.

In one or more embodiments, the method of using the nanocomposite compositions can include, but are not limited to: a rheological modifier, viscosifier, spreadability modifier, “skin feel” modifier, stabilizer, colorant, topical cosmetic, facial cleanser, hair cleanser, body cleanser, deodorant agent, skin toner, toxin remover, skin revitalizer, anti-aging or anti-wrinkle agent, chemical exfoliants, anti-inflammatory agent, anti-oxidizing agent, UV-protecting agent, anti-microbial agent, astringent agent, or combination thereof. In an embodiment, the one or more nanocomposite compositions can be used as base formulation and/or a system of additives for topical cosmetic and dermal applications. In addition to serving as a potentially suitable alternative for various topical dermal applications, the components can be healthy and beneficial to consumers, which also means that the nanocomposite compositions do not require additional surfactants that may be detrimental to skin, hair, and consumer health.

The nanocomposite composition can be used in a wide variety of commercial products. For example, the commercial products the nanocomposite composition can be used in include, but are not limited to: facial cleansers, body cleansers, deodorant agent, skin toner, toxin remover, skin revitalizer, anti-aging/anti-wrinkle agents, chemical exfoliant, anti-inflammatory agent, anti-oxidizing agent, UV-protecting agent, anti-microbial agent, and/or astringent agent. In addition to serving as a potentially suitable alternative for various topical dermal applications, the components can be healthy and beneficial to consumers, which also means that the nanocomposite compositions do not require additional surfactants that may be detrimental to skin, hair, and consumer health.

From the rheological study, it is evident that the nanocomposite compositions form network structures with viscoelastic properties that are desirable for applications in cosmetics. These materials exhibit a yield stress and a shear-thinning rheology. This can allow for the material to be processed easily with a lower viscosity under high shear conditions, but will allow for shelf-stability under low shear or “zero-shear” conditions. When applied on the skin by the end-user, the shear thinning properties will allow the material to be spread easily, but will help keep the cosmetic material in place when no shear force is applied.

This study investigates rheological properties of novel, algae-based nanocomposites developed for potential use in dermal applications. The purpose of this investigation is to develop a highly tunable and completely biocompatible cosmetic base the nanocomposite composition and demonstrate how rheology relates to spreadability in its application. It was hypothesized that Spirulina biomass could be used in conjunction with additions of titania (TiO₂) nanoparticles and crosslinking and binding agents of calcium and/or citric acid to form nanocomposite materials with highly tunable rheological characteristics in absence of chemical surfactants. In this study, Arthrospira platensis (Spirulina) biomass, titanium (IV) dioxide, calcium chloride, and citric acid are used to develop a the nanocomposite composition that is then tested for a wide range of rheological characteristics and sensory-related properties. The rheological tests include amplitude sweep, frequency sweep, viscosity flow curve, 3-interval strain oscillatory thixotropy, and creep—recovery. Additionally, yield stress, complex viscosity, zero-shear viscosity, and power law indices were determined. The sensory-related properties that are discussed include formulation pH, static load spreadability, and dynamic shearing spreadability. After completing the investigations, it was concluded that the surfactant-free nanocomposite compositions were highly tunable, in terms of viscosity, rigidity, and thixotropy, when varying additive concentration during process steps.

A sustainable, biocompatible algae-based nanocomposite was investigated for the purpose of developing a surfactant-free the nanocomposite compositions that is simultaneously beneficial to dermal health and environmentally safe. Rheological and sensorial properties of nanocomposite compositions were determined for the characterization of algae-based nanocomposite formulations for its rheological tunability and spreading characteristics as a topical cosmetic formulation. The study is the first look into the rheological and sensorial properties of this algae-based material as a cosmetic formulation and its tunable capabilities with additive concentration. Spirulina biomass can have a significant potential as a major constituent of cosmetic formulations, which could initiate the makings of a new market of cooperation between the algae cultivation industry and the beauty/wellness industries.

For consumers, texture and sensory perception of topical products is a complex process that plays a significant part in the acceptance and overall demand of the product [15,16]. These sensory properties of dermal products are often characterized by the material pH and rheological profiles. Additionally, the analysis and understanding of the rheology of the nanocomposite compositions can make it possible to optimize manufacturing processes, control product quality, and maintain storage stability [17,18]. The structural strength of the nanocomposite composition can be rheologically evaluated with an oscillatory measurement of amplitude sweep, which can measure yield stresses and many viscoelastic properties. A material's structural strength is a crucial parameter for process manufacturing, extraction from storage, as well as application to topical surfaces (e.g. skin, hair, etc.) [19]. The flow behavior and spreadability of topical products can be rheologically evaluated by shear viscosity measurements [15,20]. Typical cosmetic formulations depict shear-thinning behavior, where viscosity continues to decrease exponentially as shear rate increases. Often employed for food and cosmetic products, transient rheological measurements such as creep and thixotropy are advanced measurements that characterize a material's structural response and regeneration after structure decomposition, which is important for optimizing both process design and consumer requirements. Creep-recovery measurements can be used to measure a material's response to constant stresses, structure regeneration after the constant stress, and to calculate the zero-shear viscosity of a material. Oscillation thixotropic measurements can be used to measure the material response to both low and high strain constants, as well as the structure regeneration after high strain.

In pursuit of low-cost, renewable, and biocompatible nanocomposite composition for cosmetic applications, this study focuses on characterizing novel algae-based nanocomposite composition by elucidating their sensory and rheological properties that are dependent on different additives and their concentrations. The nanocomposite compositions are centered around food-grade Arthrospira platensis biomass as a water-soluble base material to be incorporated with titanium (IV) dioxide nanoparticles, calcium chloride, and citric acid. In addition to the industries of cosmetics and pharma, Spirulina algae also has applications in biofuel production [21], wastewater treatment [22], and nutraceutical production [23]. Viscoelastic moduli, flow curves, thixotropy, yield stress, zero-shear viscosity, pH, material composition, and spreadability measurements are investigated to characterize the topical rheology and tunability of these algae-based nanocomposites.

As it currently stands, the world production of Spirulina for consumption is expected to reach 68,000 tons by the year 2025 [24]. Due to its benefits in supplementation, Spirulina algae is often considered a superfood as a result. However, Spirulina also offers many benefits to dermal applications as well. Phycocyanin, a blue photosynthetic pigment-protein complex present in Spirulina, is a natural antioxidant in reducing lipid peroxidation [25] and anti-inflammatory agent that helps in reducing skin aging [26]. This organic material also contains amino acids (e.g. glycine, proline, etc.) that support skin firmness by supporting the body's collagen production via increase in growth factors in dermal fibroblast cells [27]. In terms of loading capabilities, Spirulina contains both hydrophilic [28] and lipophilic [29] compounds, which indicates that it can be loaded with both water-based and oil-based compounds in pursuit of different desired properties of the nanocomposite composition. Furthermore, Spirulina is employed for heavy metal anti-toxicity agents [30], anti-fungal agents [31], and anti-bacterial agents [32], all of which can benefit one or more skin conditions such as candida [31], acne [32], psoriasis [33], or eczema [34].

Titanium dioxide is widely used as a pigment additive [35] in various paints, cosmetics, and plastics, while also aiding in protection from ultraviolet (UV) light [36] via topical applications (e.g. fabric, sunscreen, etc.). This inorganic oxide also has anti-microbial properties [36,37], is associated with improving skin tone and reducing blemishes [38] and enhances the longevity of products (e.g. food, pharmaceuticals, supplements, etc.). Furthermore, the FDA has permitted use of titanium dioxide in food [39], drug/cosmetics [40], personal care, and sunscreen.

Calcium directly benefits the outermost layer of skin (epidermis) by improving barrier function repair and the skin's homeostasis process [41], where the process of cell-division and cell-loss on the skin is compensated [42]. Supplementing the calcium levels in the epidermis plays a significant role in reducing the aging process by improving the proliferation of keratinocytes [43]. In cosmetic applications, calcium is often used as a viscosity agent and astringent additive, which is intended to yield a tightening or tingling sensation on the skin [44]. The FDA considers calcium and calcium chloride (CaCl₂) to be safe for food and dermal applications [45,46]. It is important to note that calcium chloride is also considered to be an effective preservative [47] of foods and personal care products [48], which is also applicable for preserving Spirulina-based compounds [49].

In addition to being a natural preservative [50,51], citric acid has been used in cleaning formulations for both toxin removal and bacterial disinfectant, which includes issues with acne and infections [52-54]. For cosmetic applications, it is used as a chemical exfoliant by forming alpha hydroxy acids [55] and it is also effective in lightening the pigment of the skin [56,57]. Additionally, citric acid facilitates improved absorption of minerals/metals [58] and revitalizes the skin via skin tissue regeneration process improvements [59-61] by acting as an antioxidant [62] where lipid peroxidation [63] and inflammation are decreased by reducing cell degranulation and attenuating the skin's release of inflammatory compounds [64], which reduces the skin's aging process [65]. Citric acid is regarded as a “generally safe” compound by the FDA for food [66,67], cosmetic [68,69], and drug formulations[70].

### Examples

To provide a better understanding of the foregoing discussion, the following non-limiting examples are offered. Although the examples can be directed to specific embodiments, they are not to be viewed as limiting the invention in any specific respect.

### Materials and Reagents

Spirulina blue-green algae biomass was purchased from Acetar Bio-Tech Inc. (Xi'an, China) with a protein content of 62.2 wt %. Titanium dioxide (TiO₂) nanoparticles (rutile, 100 nm, 99.9% purity) was purchased from US Research Nanomaterials, Inc. (Houston, TX, USA). Calcium chloride dihydrate (CaCl₂·2H₂O) and anhydrous citric acid were purchased from Innovating Science (Avon, NY, USA). For the purpose of this study, the calcium chloride dihydrate and anhydrous citric acid were each used to make one solution of 10 wt % calcium and one solution of 10 wt % citric acid. All formulations use deionized water.

### Sample Preparation of Nanocomposites

To make samples the nanocomposite compositions, deionized water and the 10 wt % calcium solution are first mixed together, followed by titanium dioxide (TiO₂) nanoparticles and Spirulina powder, while the 10 wt % citric acid solution is always added last. However, the composition of the four additives is investigated systematically in terms of initial formulation: Spirulina is investigated for concentrations of 0 wt %, 4.0 wt %, 8.0 wt %, and 12.0 wt %, while titanium dioxide nanoparticles are investigated for 7.5 wt %, 10.0 wt %, 12.5 wt %, and 15.0 wt %. The calcium additive is investigated for concentrations of 1.5 wt %, 3.0 wt %, 4.5 wt %, and 6.0 wt %. Due to its cross-linking and chelating properties, the citric acid additive is investigated last and at concentrations of 0 wt %, 0.25 wt %, 0.50 wt %, and 1.0 wt %. To communicate the data and rheological trendlines effectively, the nanocomposite composition containing TiO₂, algae, and calcium will be loosely labeled with the acronym of TAC, while the nanocomposite composition containing TiO₂, algae, calcium, and citric acid will be loosely labeled with TACC. It is important to note that: a formulation labeled only as “TAC” has the base composition of 10 wt % TiO₂, 8 wt % algae, and 3 wt % calcium. Any variation from the base “TAC” formulation will be communicated in the data and listings.

When adding the necessary amount of algae material and nanoparticle, they are carefully added into the water separately and the stir plate's rotational speed is closely monitored and adjusted as necessary to avoid initial material agglomeration, loss of fluid, and minimization of foaming. Each blend is initially mixed for 15 minutes, followed by ultrasonication for 15 minutes, and then mixed again for another 30 minutes. After the blend is thoroughly mixed and sonicated, it is transferred into trays with ˜2.50 g samples in each tray. The trays are then heated in an oven at 70° C. for 1 hour. After heating, the samples are removed from the oven and allowed to cool to room temperature for 15 minutes before measurements commence. Due to the heating process, there are concentration changes because of water loss, which is tracked and listed in table data.

### Glass-Plate Spreadability Tests

Spreadability experiments in this study were conducted using glass plates and samples of 0.5 grams each. For each test, a weight was placed on top of the glass so that a 500-gram force was being applied. The weight is applied to the plates for 5 minutes and the diameter of the spread is recorded. All spreadability measurements are tested three times and an average is taken.

In preparation for spreadability measurements, the glass plates are thoroughly rinsed and dried. After each measurement, the glass plates are washed with water and minimal soap, before being dried and allowed to sit for a minimum of 15 minutes before the next measurement.

### pH Measurements

The characterization for formulation pH was carried out using an Oakton pH 6 Acorn Series Meter. The pH levels of cosmetic and dermal products have a strong impact on skin health and the sensory comfort of the user. In particular skin is naturally acidic and surfactant or alkaline formulations often damage and dry out the skin's acid mantle film, while acidic formulations strengthen and maintains its function. Once each nanocomposite was prepared, the probe was immersed into the material and the pH value was recorded after the displayed value on the pH meter remained constant for a minimum of 10 minutes. All pH measurements are tested and averaged between three iterations.

In preparation for pH measurements, the measurement probe was calibrated with three calibration buffer solutions. In between each calibration and after calibration is finished, the probe was thoroughly cleansed with water and dried. After each measurement, the probe was thoroughly rinsed with water and immersed in a beaker of clean water until the next sample was ready to be tested.

### Rheological Characterization

Oscillatory and rotational rheology characterizations were carried out on an Anton Paar Modular Compact Rheometer 302 (MCR-302) using the parallel plate method and Rheoplus software. The stainless steel parallel-plate attachment had a diameter of 25 mm. The plate gap was set to 0.1 mm for all experiments except for strain oscillatory thixotropy and creep-recovery, which were set at 1 mm. In addition to using less material per measurement, the small plate gap of 0.1 mm makes it significantly less likely to expel material during rheology experiments, allowing for testing in higher shear rate conditions. All measurements were performed at 20° C. under atmospheric pressure, and the oscillatory thixotropy measurements also have measurements performed at 70° C.

In preparation for rheology measurements, the rheometer parallel plates are initially cleaned with water, wiped with 99% isopropanol alcohol, and allowed to dry for 30 minutes. The stage temperature is set to 20° C. for all rheology experiments. Each test used a new sample to avoid hysteresis effects. The rheometer is cleaned after each experiment with water, 99% isopropanol alcohol, and allowed to dry for a minimum of 30 minutes.

### Oscillatory Rheology—Amplitude and Frequency Sweeps

Spreading properties can be described by characterizing a material's rigidity, strength, and relating contributions of elastic and viscous behavior through structural and viscoelastic measurements. Properties involving rigidity and yield stress are particularly crucial in situations leading up to the application of high shear spreading regimes.

The oscillatory linear viscoelastic measurements were comprised of the amplitude sweep and frequency sweep tests. The amplitude sweep measurements were taken in the strain range from 0.01-1,000% at a constant frequency of 1 rad/s. In particular, an advanced version of amplitude sweep is performed in this study to obtain yield stress while measuring the linear viscoelastic range (LVER), storage moduli, loss moduli, and to be able to choose a constant strain value that does not destroy the material's structure during frequency sweep experiments. Frequency sweep measurements were performed in the range from 0.1-100 rad/s at a constant strain value of 0.05%, which was determined from the amplitude sweep results. Frequency sweep measures the same viscoelastic responses as a result of angular frequency and also measures the complex viscosity of the materials.

### Rotational Rheology—Viscosity Flow Curves

For topical cosmetics and dermaceutical applications, high-shear viscosity tests are performed in order to mimic the high-shear regime when spreading products on the skin. To evaluate the non-Newtonian behavior of these materials, the viscosity flow curves with controlled shear rate (CSR) are measured in the range increasing from 1-10,000 s⁻¹. Power law indices “n” and “k” were determined from experimental data by evaluating viscosity as a function of shear rate as a linear trendline on a log-log graph for the 1,000-10,000 shear range, which encompasses both high-shear rates within topical applications and high-shear rates regarding manufacturing processes. The flow indices were determined by correlating the power law trendline with the power law equation for viscosity (1) and solving for “n” and “k” for each formulation.

$$η = k \cdot {\dot{γ}}^{n - 1}$$

### Transient Rheology—Thixotropy and Creep Recovery

Thixotropy and creep-recovery are often mandatory tests for pharmaceutical creams and other topical cosmetic products, which is used to characterize and understand the internal structure of the nanocomposite compositions. The oscillatory thixotropy experiments are used to evaluate a material's rigidity under low-strain conditions, as well as the time it takes to rebuild the composite structures after experiencing high-shear loads. The creep-recovery test is used to describe the materials flow under low-stress conditions, determine the structure rebuild time, and calculate zero-shear viscosity. The rebuild percentages for the oscillatory thixotropy are described for the oscillatory thixotropy in equations (2) and (3), while the rebuild percentages for the creep-recovery tests are described in equations (4) and (5). Furthermore, zero-shear viscosity values can be calculated from equation (6) by using the data in the creep-recovery test. The subscript of “recovery” depicts the amount of the storage modulus (G′) or strain (γ) that has been successfully recovered after the material's disruption in structure. The subscript of “load” and “peak” depict the storage moduli of the load-interval for the oscillatory thixotropy tests and the highest strain of the creep-interval for the creep-recovery tests, respectively. Zero-shear viscosity is represented by η₀, τ represents the constant stress that is used during each creep-recovery tests, and the

$$\frac{{t}_{2} - {t}_{1}}{{γ}_{2} - {γ}_{1}}$$

variable represents the linear regime between strain and time that occurs during the later portions of the creep-interval for the nanocomposite composition sample being tested.

$$Strain 3 ITT : {\%}_{recovered} = ( \frac{{G}_{recovery}^{′}}{{G}_{load}^{′} - {G}_{breakdown}^{′}} ) * 100$$

$$Strain 3 ITT : {\%}_{unrecovered} = ( \frac{{G}_{load}^{′} - {G}_{recovery}^{′}}{{G}_{load}^{′} - {G}_{breakdown}^{′}} ) * 100$$

$$Creep : {\%}_{recovered} = ( \frac{{γ}_{peak} - {γ}_{recovery}}{{γ}_{peak}} ) * 100$$

$$Creep : {\%}_{unrecovered} = ( \frac{{γ}_{recovery}}{{γ}_{peak}} ) * 100$$

$$Zero shear viscosity : {η}_{0} = τ * \frac{{t}_{2} - {t}_{1}}{{γ}_{2} - {γ}_{1}}$$

The first thixotropy interval and third thixotropy interval both have a constant strain rate of 1%. The first interval was set for ˜10 minutes and the third interval was set to 15 hours during 20° C., but only ˜2 hours for the 70° C. measurements. The second thixotropy interval is set to a strain rate of 100% for 5 minutes, which will thoroughly disrupt the initial structure of the material and allow the structure to rebuild during the third interval. The creep intervals were all performed at a constant stress of 50 Pa for ˜5.3 hours and the recovery intervals were maintained at a constant stress of 0 Pa for 15 hours.

### Nanocomposite Compositions

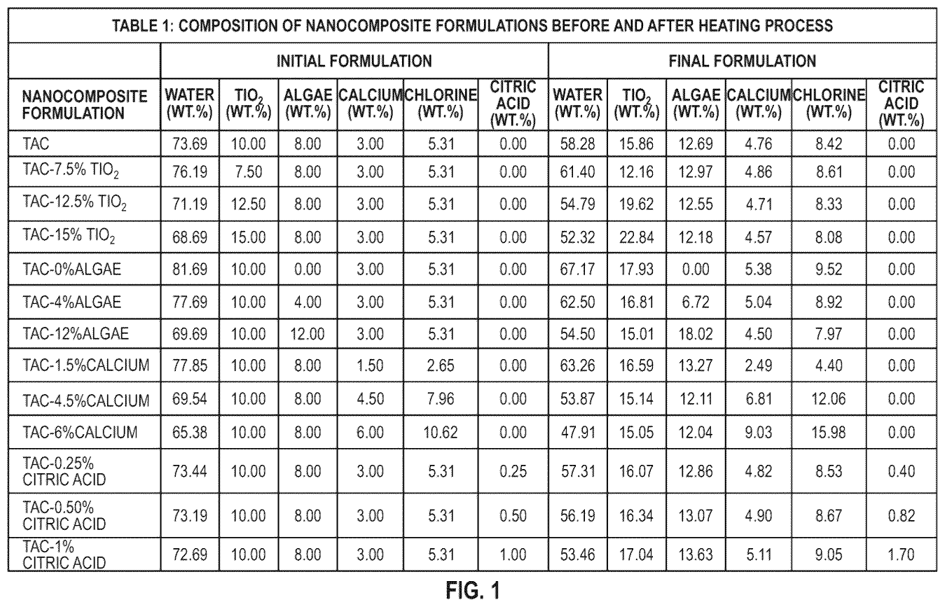

The changes in the nanocomposite compositions before and after heating are shown in Table 1 in FIG. 1. The TAC TiO₂/algae/calcium percentage ratio changed from 10/8/3 to 15.86/12.69/4.76, which is due to the loss of water from the heating step of the process. As a result of this water loss, all nanocomposite compositions investigated will have a similar composition change as the base TAC formulation does. For the variety of nanocomposite compositions measured, the maximum water content was calculated at 67.17 wt % and the minimum water content was calculated at 47.91 wt % (see column 8 of Table 1).

Based on observation, the TAC formulations with fluctuations of either 7.5% TiO₂, 0% algae, 4% algae, or 1.5% calcium remained relatively more liquid-like after sample preparation, which indicates that an adequate amount of TiO₂, algae, and calcium are necessary components to make semi-solid products with desirable textures and strengths. Combining this observation with the composition data seen in Table 1, it can be inferred that optimal semi-solid products for these materials must not exceed ˜60.0 wt % water.

### Glass-Plate Spreadability of Nanocomposite Compositions

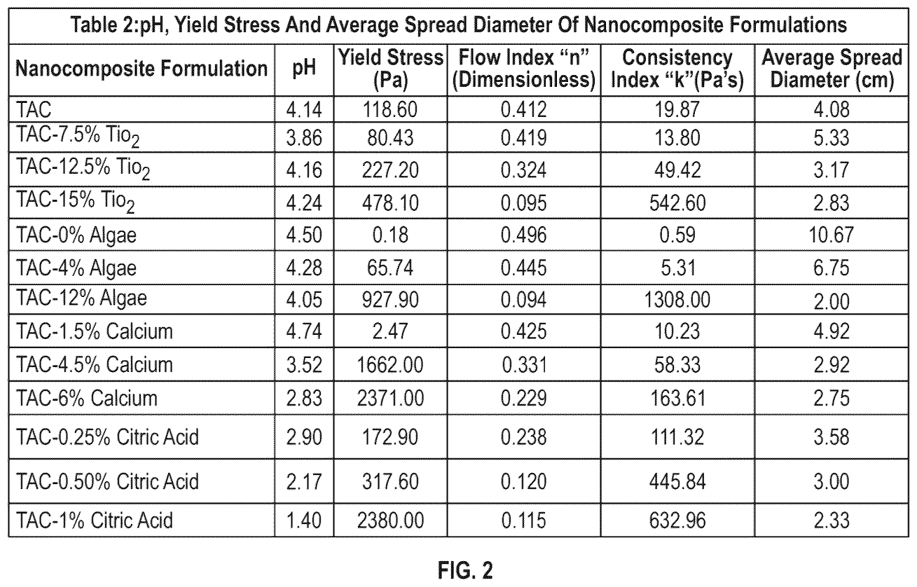

The average spreadability diameter results of the nanocomposite composition are listed in column 6 of Table 2 of FIG. 2. All of the nanocomposite compositions displayed reduced spreading diameter, under a static force, as the composition of additives increased and composition of water decreased (reported in Table 1). The nanocomposite compositions of varying TiO₂ concentration displayed spread diameters from 2.83-5.33 cm, while the varying algae formulations displayed spread diameters from 2.00-10.67 cm. Calcium variations displayed spread ranges from 2.83-4.92 cm and citric acid varying formulations displayed ranges from 2.33-3.58 cm. The largest average spreadability diameter was exhibited as 10.67 cm by the formulation “TAC—0% Algae,” while the lowest average spreadability diameter was exhibited as 2.00 cm by the “TAC—12% Algae” formulation.

### pH of Nanocomposite Compositions

Column 2 of Table 2 shows the pH changes as additive composition is varied, respectively. As the concentration in TiO₂ increased, the formulation pH also increased in the range from 3.86-4.24. However, for increasing concentrations of algae, calcium, and citric acid, the nanocomposite composition pH decreased in the ranges from 4.50-4.05, 4.74-2.83, and 4.14-1.40, respectively. The highest nanocomposite composition pH value achieved of 4.74 was “TAC-1.5% Calcium” and the lowest value of 1.40 was achieved with “TACC—1% Citric Acid.”

### Oscillatory Rheology

### Amplitude Sweep and Yield Stress

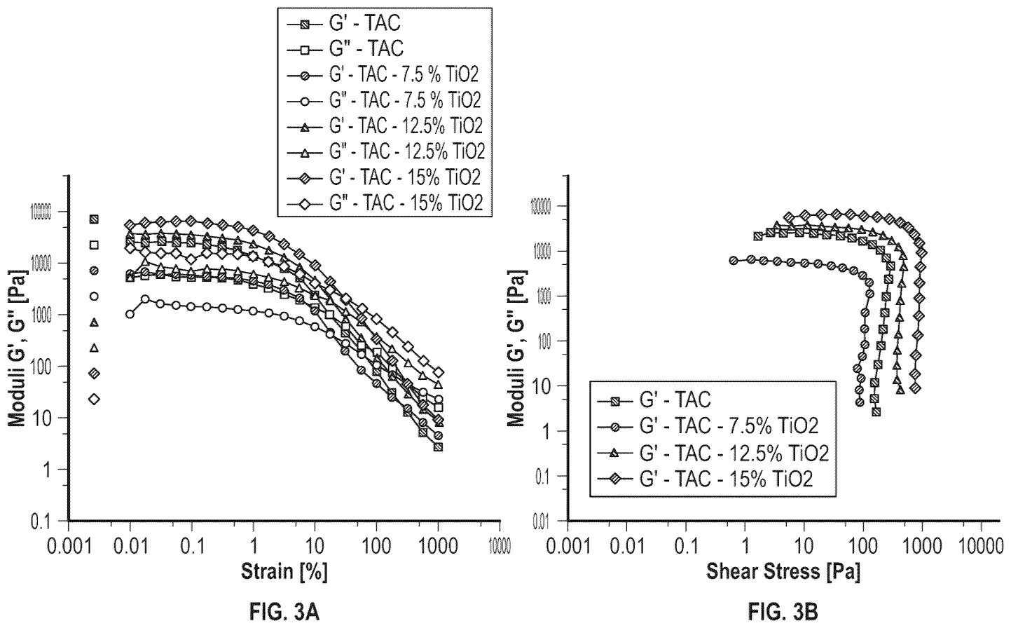

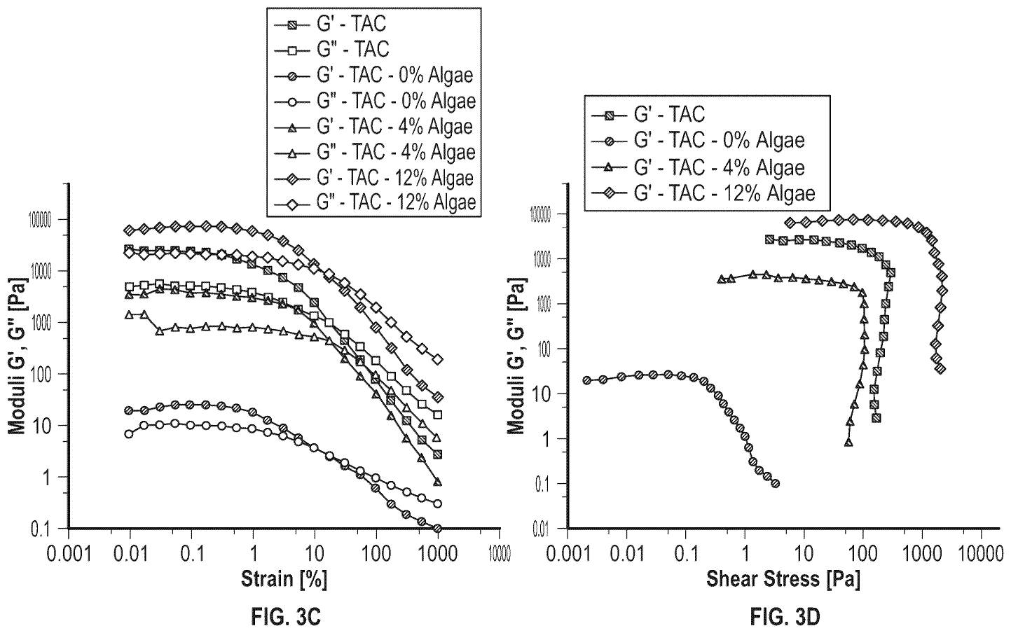

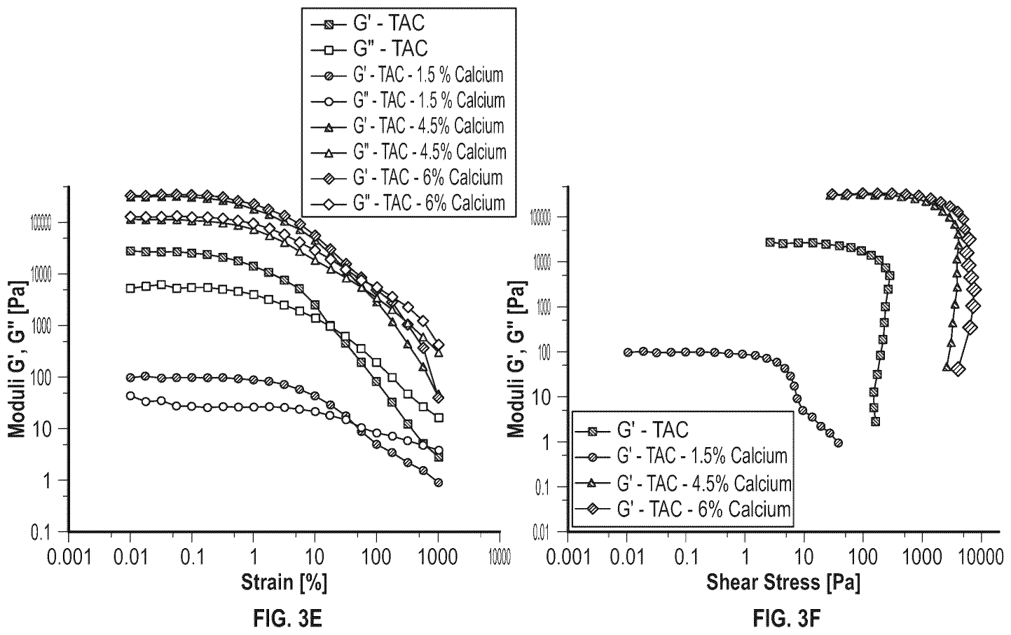

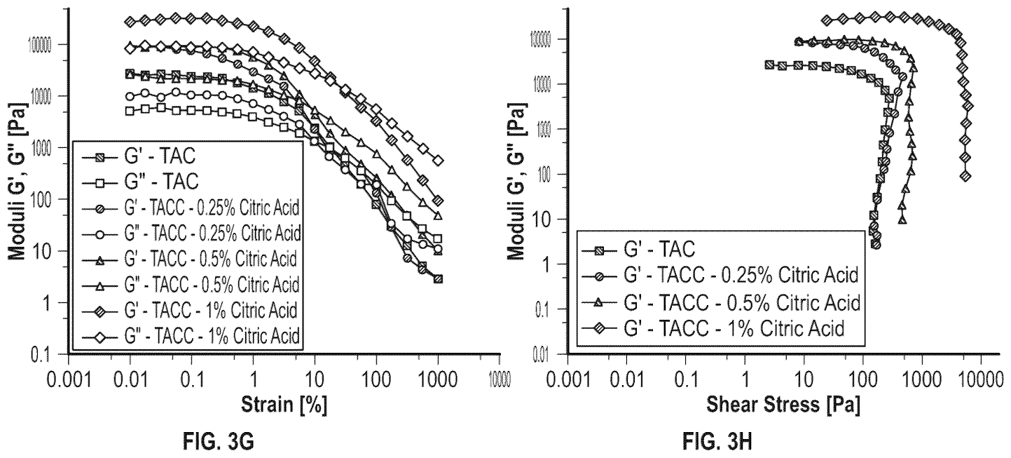

As shown in FIGS. 3A-3H, all nanocomposite compositions were tested in a frequency-controlled amplitude (strain) sweep at a constant temperature of 20° C. and a constant frequency of 1 rad·s⁻¹. The LVER for all nanocomposite compositions investigated was approximately between 0.01-1% strain. Within the LVER, all nanocomposite compositions exhibited storage moduli that were greater than the loss moduli, which indicates that their viscoelastic properties are elasticity dominated. Additionally, all nanocomposite compositions displayed increases in moduli and yield stress (column 3 of Table 2) as their respective additive concentrations increased. The nanocomposite compositions with the highest magnitudes in moduli are “TAC—6% Calcium”, “TAC—4.5% Calcium”, and “TACC—1% Citric Acid” with values of 2.82-10⁵ Pa, 2.74-10⁵ Pa, and 2.31-10⁵ Pa, respectively. However, the nanocomposite compositions with the lowest moduli magnitudes are “TAC—1.5% Calcium” and “TAC—0% Algae” with values of 131 Pa and 23 Pa, respectively.

Furthermore, all storage and loss moduli crossover points were measured to be above the 10% strain. The crossover points of the nanocomposite compositions exhibited distinct deviations as result of changing either TiO₂ or calcium concentration, whereas there were no consistent deviations as a result of varying algae or citric acid concentration. The ranges of observed yield stress for the measured variations of TiO₂, algae, calcium, and citric acid are 80.43-478.10 Pa, 0.18-927.90 Pa, 2.47-2371.00 Pa, and 118.60-2380.00 Pa, respectively. The formulation with the lowest yield stress of 0.18 Pa was “TAC—0% Algae,” while the highest yield stress of 2380.00 Pa comes from the “TACC—1% Citric Acid” formulation.

### Frequency Sweep and Complex Viscosity

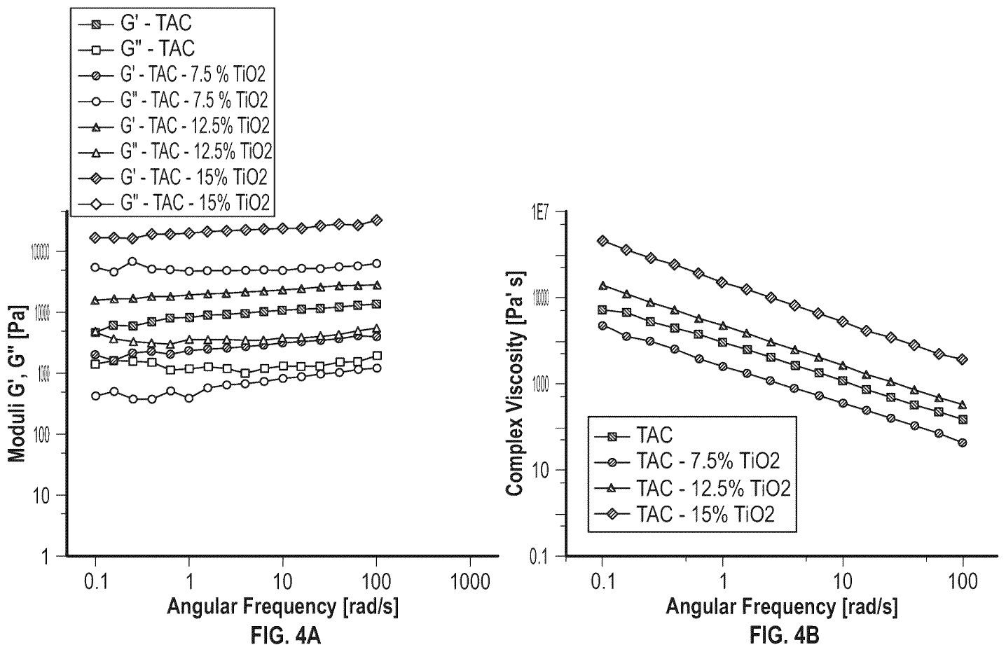

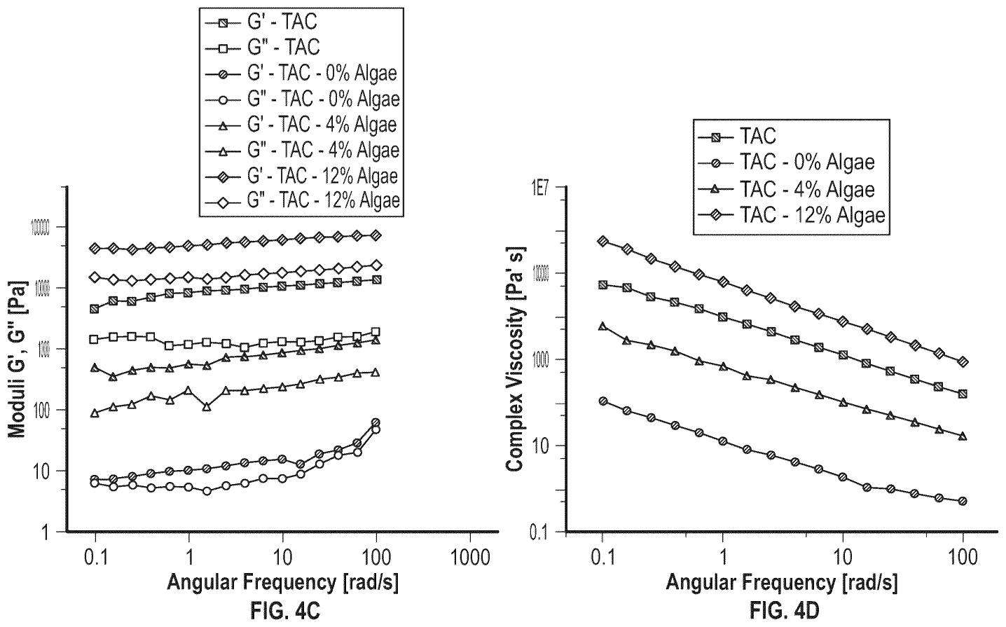

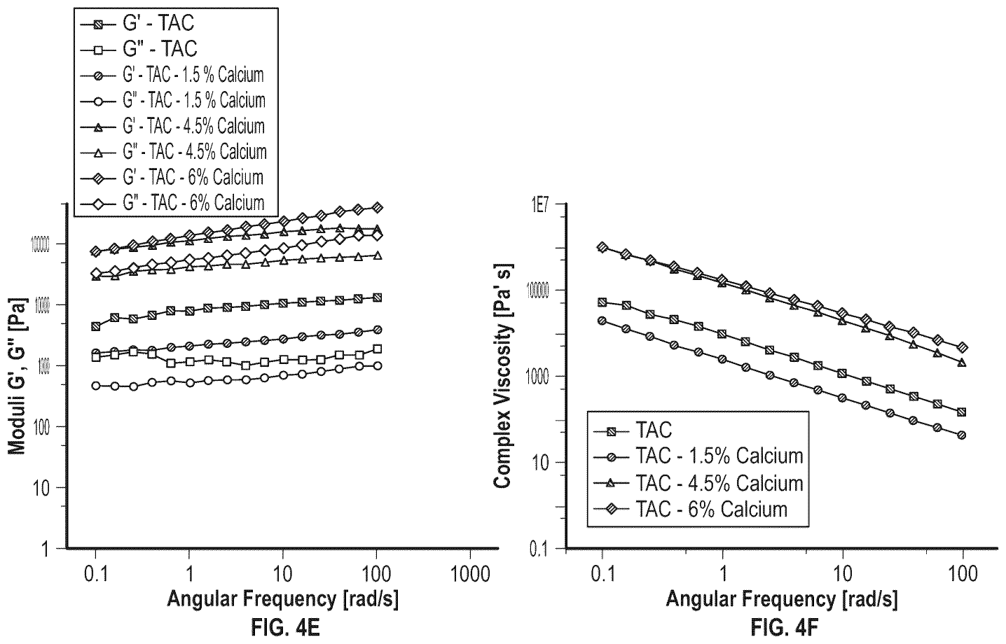

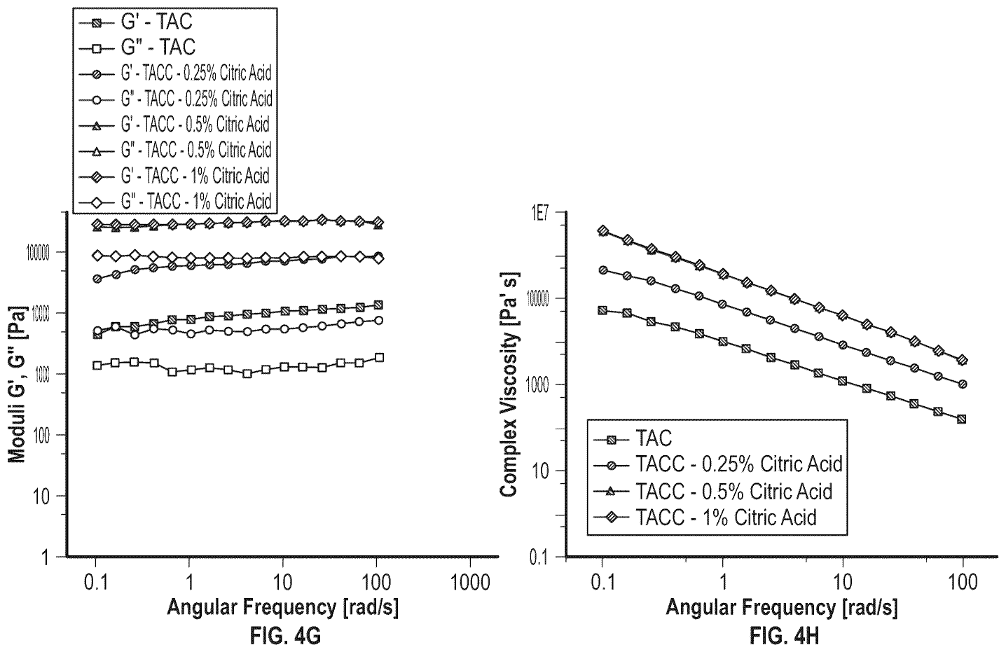

As shown in FIGS. 4A-4H, all nanocomposite compositions were tested in a strain-controlled frequency sweep at a constant temperature of 20° C. and a constant strain of 0.05%. Throughout the entire investigated angular frequency range of 0.1-100 rad/s, all nanocomposite compositions display storage moduli that are greater than loss moduli, which serves as further evidence that the viscoelastic properties are structurally elastic. Unlike the amplitude sweeps, the frequency sweep responses did not yield any crossover point for any nanocomposite composition that was investigated. Like the amplitude sweep responses, all nanocomposite compositions displayed increases in moduli and complex viscosity values as their additive concentrations increased. The highest frequency sweep moduli values are 2.95·10⁵ Pa and 2.80·10⁵ Pa from the “TACC—1% Citric Acid” and “TACC—0.5% Citric Acid” formulations, respectively. The lowest frequency sweep moduli values are 548 Pa and 10.5 Pa from the “TAC—4% Algae” and “TAC—0% Algae” nanocomposite compositions, respectively. The complex viscosity values for all nanocomposite compositions were approximately equivalent to their storage modulus values measured.

### Rotational Rheology

### Flow Curves

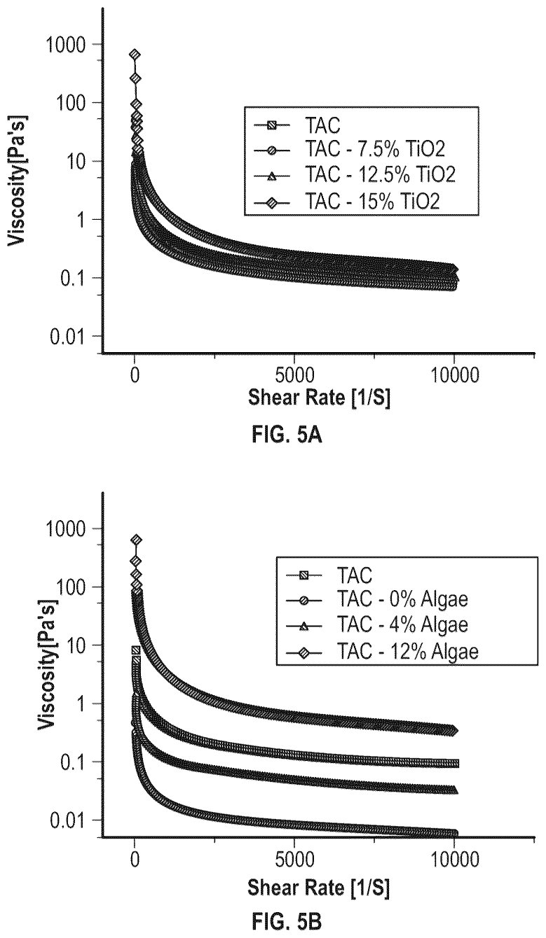

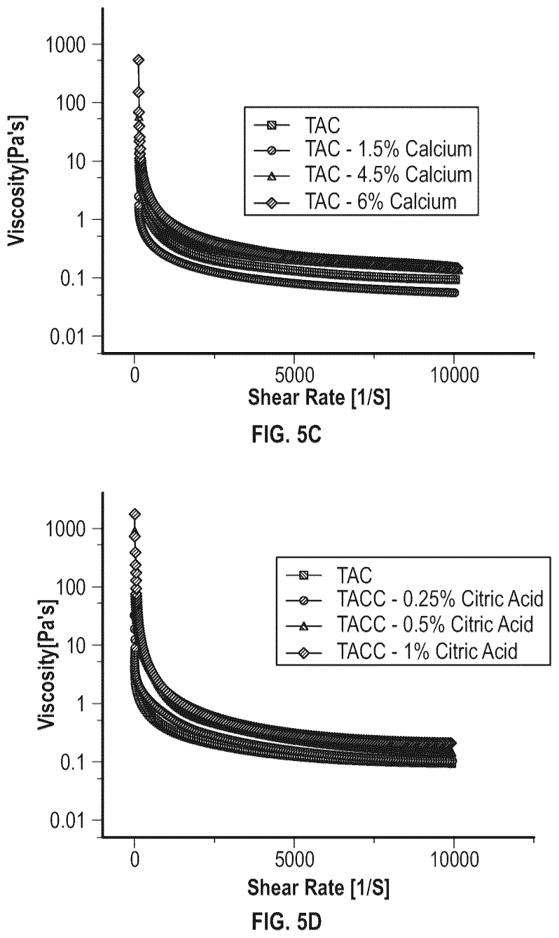

As shown in FIGS. 5A-5D, all nanocomposite compositions were tested in a controlled-shear rate flow curve at a constant temperature of 20° C. All nanocomposite compositions exhibited shear-thinning behavior in the 1-10,000 s⁻¹ shear range that was investigated. Increasing the concentration of each varied additive also increased the viscosity of the material. The most viscous nanocomposite compositions investigated was “TAC—12% Algae”, followed by “TACC—1% Citric Acid”. However, the least viscous nanocomposite composition was “TAC—0% Algae”, followed by “TAC—1.5% Calcium”. Compared to the other additives, the concentration of Spirulina displays the widest changes in viscosity trends.

The power-law flow (n) and consistency (k) indices for the shear range between 1,000-10,000 s⁻¹ of each nanocomposite composition are listed in columns 4 and 5 of Table 2, which were determined through trendline best fit with R² values between the range of 0.9856-0.9981. The effect of different additive concentrations is clear, where the flow index decreases and the consistency index increases as additive concentration increases. For the materials with varying amounts of TiO₂, algae, calcium, and citric acid, the flow index ranged between 0.095-0.419, 0.094-0.496, 0.229-0.425, and 0.115-0.412. While the varying amounts of TiO₂, algae, calcium, and citric acid had consistency index values that ranged between 13.80-524.60 Pa·s, 0.59-1,308.00 Pa·s, 10.23-163.61 Pa·s, and 19.87-632.96 Pa·s.

### Transient Rheology

### Oscillatory 3-Interval-Thixotropy

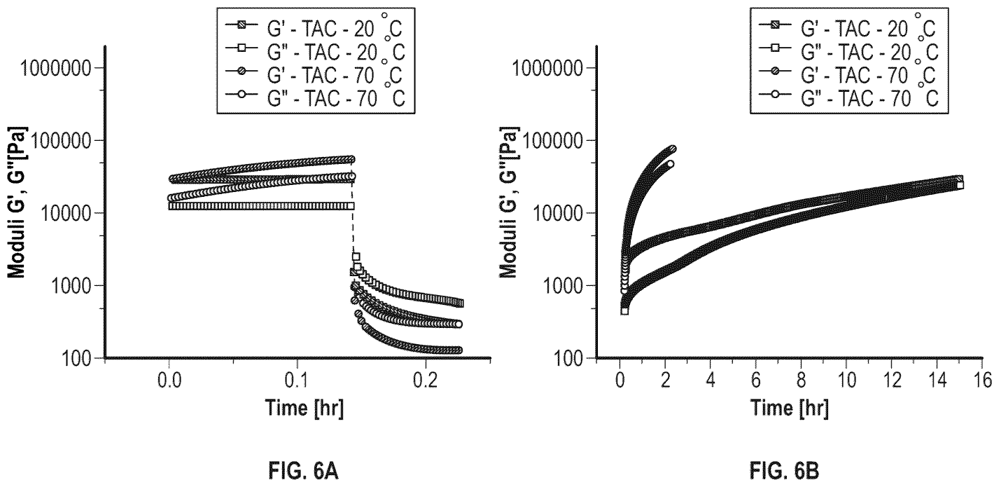

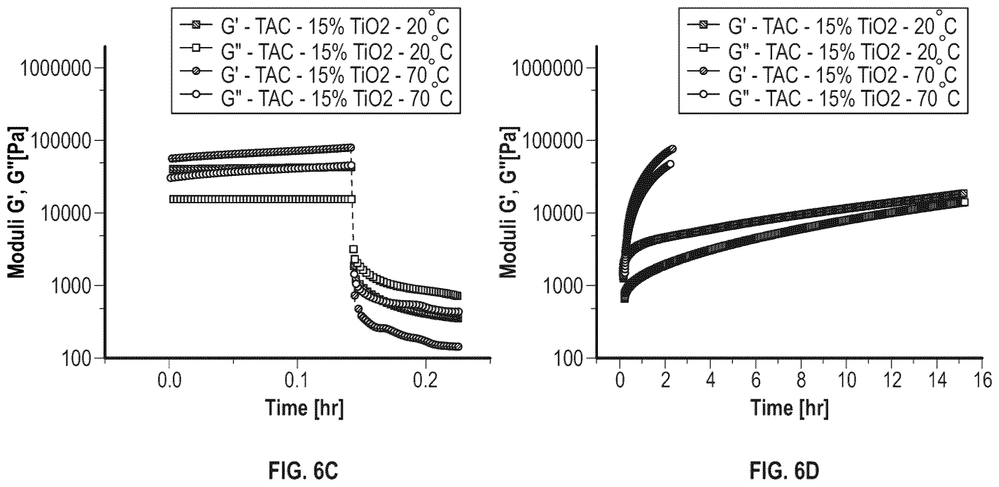

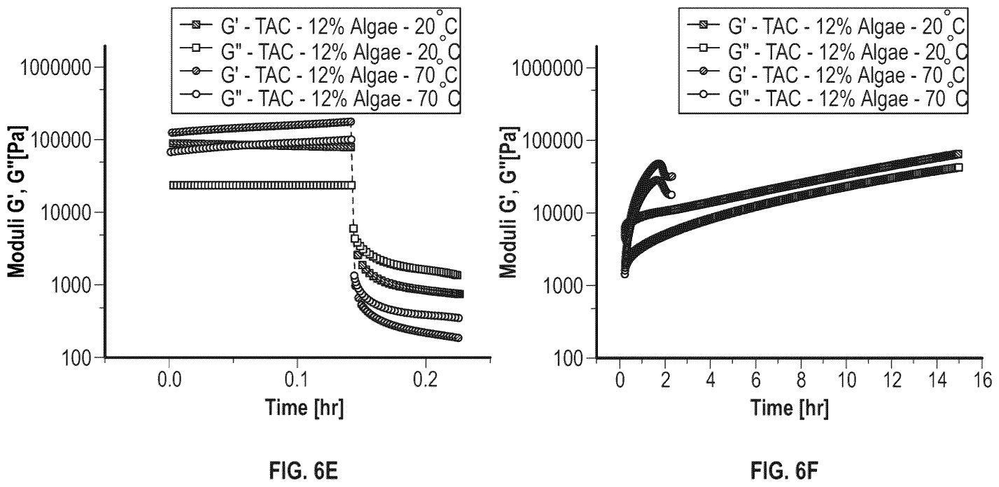

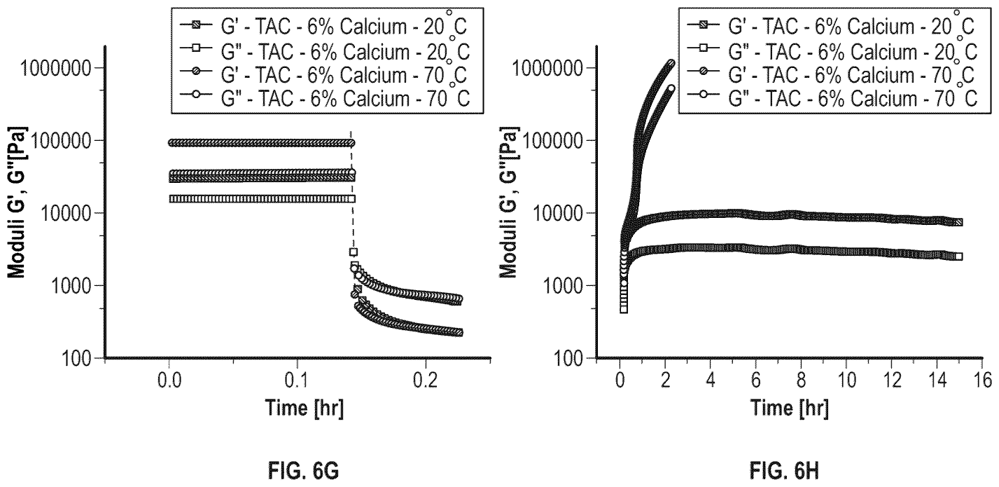

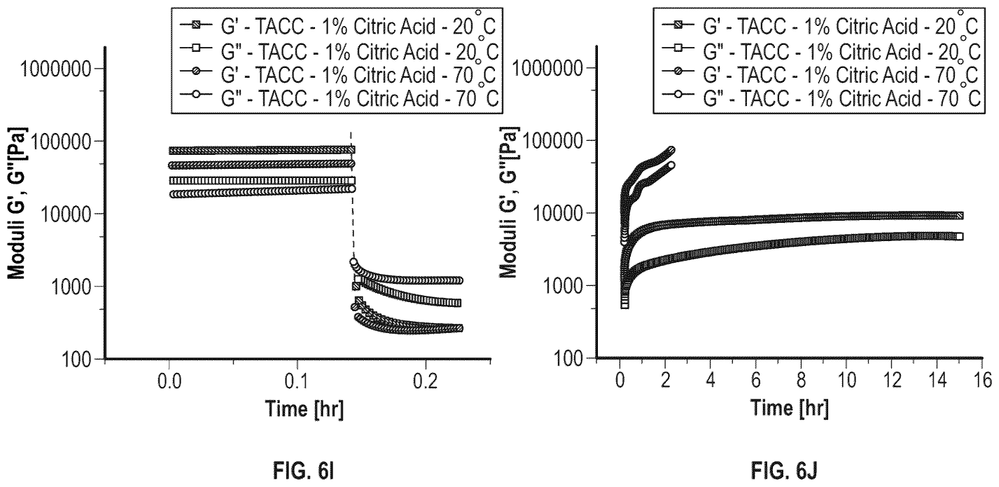

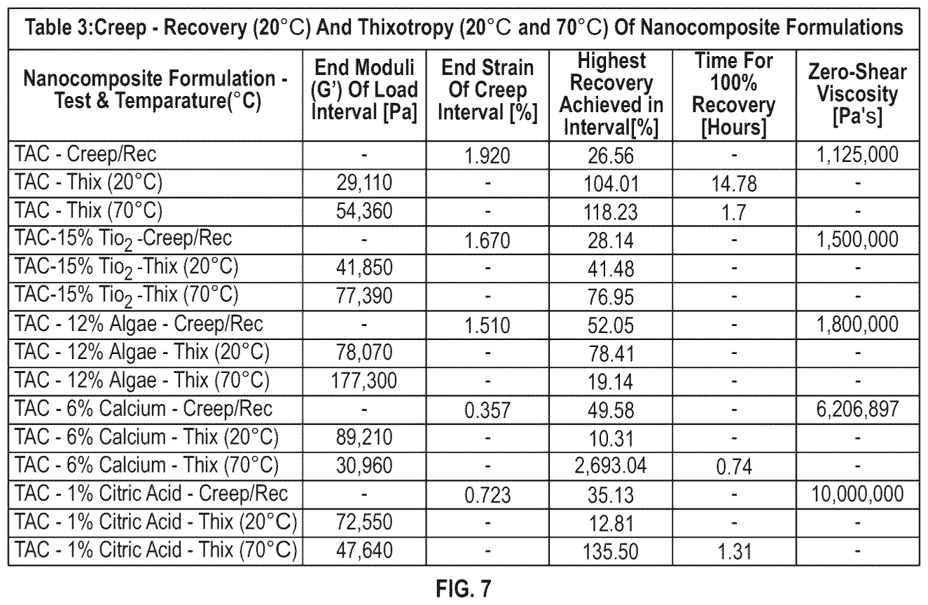

As shown in FIGS. 6A-6J, the algae-based nanocomposite compositions were investigated for oscillation strain recovery (at both 20° C. and 70° C.) in three intervals: load interval, breakdown interval, and recovery interval. For these measurements, the base TAC formulation is compared with the other nanocomposite compositions that contain the highest investigated amounts of each additive (TiO₂, algae, calcium, citric acid) in order to elucidate the changes that occur when altering each of their concentrations. Table 3 in FIG. 7 provides storage modulus values at the end of the loading phase (column 2), highest percent recovery data (column 4), and the time needed for full structural recovery (column 5), if applicable.

Previously mentioned in the methods section, the first interval is held at a constant strain of 1% in order to load and maintain the material structure, the second interval breaks the material's structure by inducing a high strain of 100%, and the third interval is held at a constant strain of 1% so that the material's structural recovery can be measured over time. At 20° C., all material structures were stable at the 1% strain loading interval, experienced significant breakdown during the 100% strain interval, and exhibited measurable amounts of recovery during the 1% strain recovery interval. However, for 70° C. measurements, three nanocomposite compositions (TAC, TAC—15% TiO₂, and TAC—12% Algae) experienced structure strengthening during the load interval and two nanocomposite compositions (TAC—6% Calcium and TACC—1% Citric Acid) experienced structure softening during the load interval. Additionally, the same three nanocomposite compositions (TAC, TAC—15% TiO₂, and TAC—12% Algae) experienced a more significant breakdown during the 100% strain interval and the other two nanocomposite compositions (TAC—6% Calcium and TACC—1% Citric Acid) experienced breakdowns that were comparable to the 20° C. conditions. All nanocomposite compositions experienced measurable structural recoveries in the 1% strain recovery interval and all formulations, with the exception of the “TAC—12% Algae” material, experienced improved structural recoveries in higher temperature conditions.

At 20° C., the TAC nanocomposite maintained a static modulus during the 1% strain load interval, followed by a reduction in structure during the 100% strain breakdown interval. However, at 70° C., the TAC nanocomposite exhibited increasing modulus values in the load interval as time passed, followed by a more significant reduction in structure during the breakdown interval at 100% strain. For the 1% strain recovery interval, the structure was rebuilt 100% successfully for both temperature conditions, although the structural recovery was faster for the 70° C. conditions. The time required to achieve 100% recovery for both the 20° C. and 70° C. conditions is 14.78 hours and 1.7 hours, respectively. Additionally, for the 20° C. and 70° C. conditions, the maximum recovery percentages observed were 104.01% and 118.23%, respectively.

Compared to the base TAC formulation, increasing the concentration of TiO₂ to formulate “TAC—15% TiO₂” yielded a small increase in modulus values during the loading interval and breakdown interval, while yielding a decrease in modulus values in the recovery interval during both 20° C. and 70° C. conditions. Within the time intervals measured, the structure for this material reached a maximum recovery of 41.48% in the 20° C. condition and 76.95% in the 70° C. condition. Thus, 100% recovery was not achieved in the time intervals that were measured, but the structural recovery was faster and more effective during the 70° C. condition.

In comparison to the base TAC nanocomposite for the 20° C. conditions, increasing the concentration of Spirulina algae to prepare “TAC—12% Algae” yielded a moderate increase in modulus values during the loading interval, breakdown interval, and recovery interval. However, the 70° C. condition exhibited large increases in modulus values for the load interval, small increases during the breakdown interval, and a significantly reduced recovery in the recovery interval. Furthermore, the recovery interval during the 70° C. condition began to decrease in modulus value and then plateau. Within the time intervals measured, the structure for this material reached a maximum recovery of 78.41% in the 20° C. condition and 19.14% in the 70° C. condition, where 100% recovery was not achieved within the measured time intervals.

Compared to the base TAC formulation regarding the 20° C. condition, increasing the concentration of calcium to achieve “TAC—6% Calcium” yielded a large increase in modulus values during the loading interval, small decrease during the breakdown interval, and a significantly reduction in the recovery interval. In contrast to these results, the 70° C. condition resulted in a minor reduction in load moduli, negligible modulus change during the breakdown interval, and substantial increases in modulus during the recovery interval. Within the time intervals measured, the structure for this material reached a maximum recovery of 10.31% in the 20° C. condition and 2,693.04% in the 70° C. condition. The time required to achieve 100% structural recovery in the 70° C. condition is 0.74 hours, while the 20° C. condition failed to achieve 100% recovery and even exhibited signs of further decreasing material strength as time passed within the recovery interval.

In comparison to the base TAC nanocomposite under 20° C. conditions, increasing the concentration of citric acid to prepare “TACC—1% Citric Acid” yielded a large increase in modulus values in the loading interval, small decrease during the breakdown interval, and significant reduction regarding the recovery interval. for the 20° C. conditions. However, the 70° C. condition exhibited a small decrease in modulus values for the load interval, negligible changes during the breakdown interval, and a slight increase in recovery during the recovery interval. For the time intervals measured, the structure for this material reached a maximum recovery of 12.81% in the 20° C. condition and 135.50% in the 70° C. condition. The time required to achieve 100% structural recovery in the 70° C. condition is 1.31 hours, while the 20° C. condition did not achieve a 100% recovery.

### Creep—Recovery

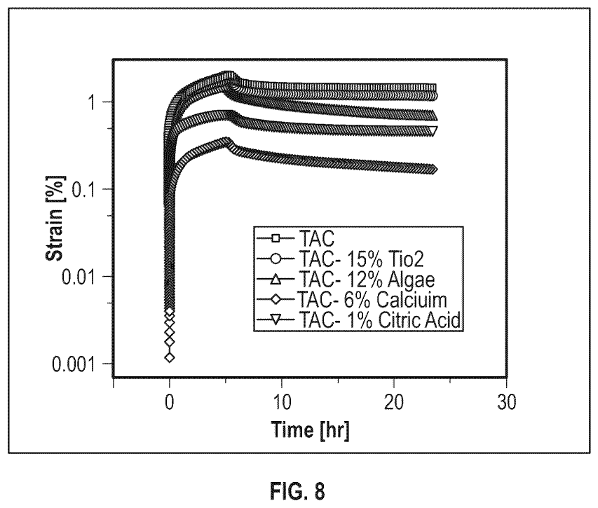

The algae-based nanocomposite compositions were investigated for constant stress response and recovery in two intervals: the creep interval and the recovery interval, as shown in FIG. 8. Like the oscillatory strain thixotropy measurements, the base TAC formulation is compared with the other formulations that are individually comprised of the highest additive concentrations (TiO₂, algae, calcium, or citric acid) that was investigated in this study, for the purpose of characterizing the changes in zero-shear viscosity and structural recoveries. Table 3 provides the strain percent values at the end of the creep interval (column 3), highest percent recovery data (column 4), the time (if applicable) needed for full structural recovery (column 5), and the calculated zero-shear viscosity for each sample (column 6).

Previously mentioned in the methods section, the first interval is held at an appropriate constant stress of 50 Pa (based on LVER data from the amplitude sweeps) in order to load and allow the material to obtain an increasing linear trend between strain percent and time, while the second interval reduces the constant stress to 0 Pa and the material's structural recovery can be measured as time passes. As concentration of each additive increased individually and compared with the base TAC formulation, the observed maximum strain value was reduced and the zero-shear viscosity was increased. The calculated zero-shear viscosities were 1.13·10⁶ Pa·s, 1.50·10⁶ Pa·s, 1.80·10⁶ Pa·s, 6.21·10⁶ Pa·s, and 1·10⁷ Pa·s for the nanocomposite compositions of TAC, TAC—15% TiO₂, TAC—12% Algae, TAC—6% Calcium, and TACC—1% Citric Acid, respectively. The maximum strain observed for TAC, TAC—15% TiO₂, TAC—12% Algae, TAC—6% Calcium, and TACC—1% Citric Acid were 1.920%, 1.670%, 1.510%, 0.357%, and 0.723%, respectively. Additionally, no material achieved 100% recovery from these tests, where the maximum recovery percentages of the TAC, TAC—15% TiO₂, TAC—12% Algae, TAC—6% Calcium, and TACC—1% Citric Acid formulations are 26.56%, 28.14%, 52.05%, 49.58%, and 35.13%, respectively. Thus, the materials with the best recovery from this tests were the “TAC—12% Algae” and “TAC—6% Calcium” nanocomposite composition.

### P-Value Correlations

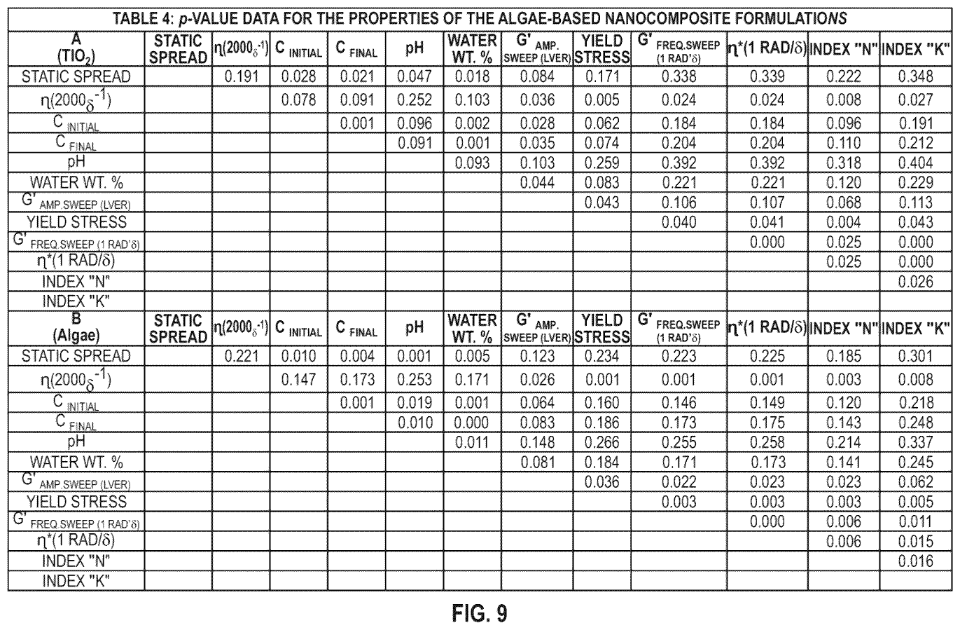

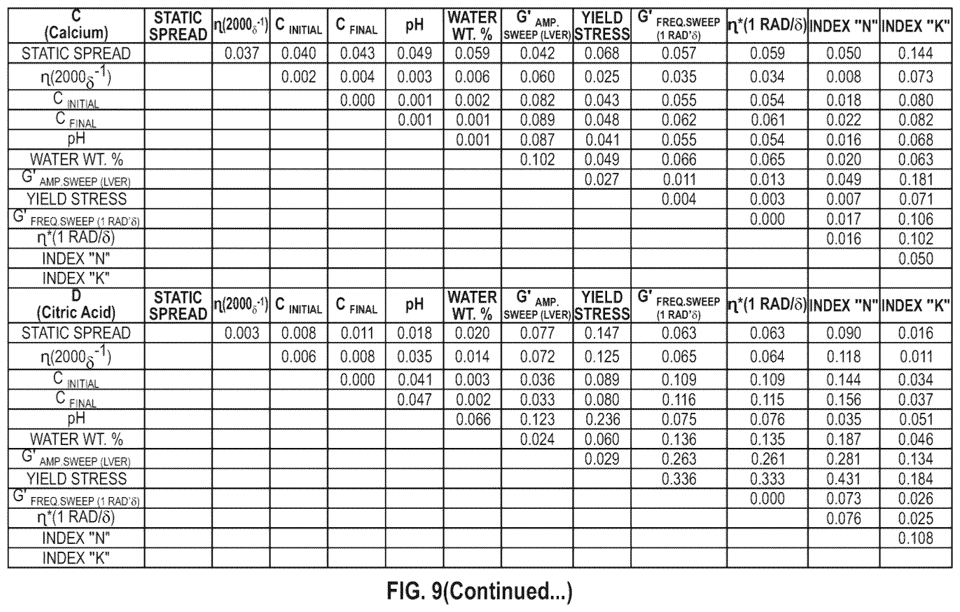

Table 4 in FIG. 9 lists the p-value-assessed statistical correlations between the relevant parameters for the nanocomposite compositions studied.

### Nanocomposite Compositions

Due to the heating process when preparing the samples, the water percentage of the nanocomposite composition is reduced and the percentage of the additives is increased, which results in more desirable semi-solid formulations that can be tuned and refined for different needs. In this process, an excess amount of water is used as medium so that the raw additives are able to disperse and interact effectively, while the heating step removes some of the excess water in order to create the different textural materials. For batch processing, the evaporated water can easily be recycled into another batch process, which would reduce water and thermal expenses.

### 4.2 Spreadability of Nanocomposite Compositions

Due to the fact that cosmetic formulations are largely designed on the basis of manual application and user sensory satisfaction, one major objective of this study is to investigate the spreadability properties of the nanocomposite compositions and how it changes with different additive concentrations. Because dermal spreadability has both sensorial and rheological differences under static and dynamic conditions, both situations were evaluated and exhibit different correlations for each material, which can be statistically analyzed using the p-value charts that are assembled in Tables 4A, 4B, 4C, and 4D. In this discussion, the static spreadability is characterized with the glass-plate measurements. However, given that all nanocomposite compositions exhibit shear-thinning behaviors, the dynamic spreadability is characterized and discussed in regards to the viscosity measurements, at which the viscosity values chosen were the measurements at 2,000 s⁻¹.

The static spreadability of the TiO₂-varied nanocomposite compositions have strong correlations with the water percentage, TiO₂ concentration, and material pH. On the other hand, the dynamic spreadability of these nanocomposite compositions have strong correlations with yield stress, frequency sweep moduli, and complex viscosity, followed by amplitude sweep moduli and both power law indices.

The most significant correlations for static spreadability with the algae-varied formulations are formulation pH, water percentage, and Spirulina concentration. However, the dynamic spreadability was most significant with frequency sweep moduli, complex viscosity, and yield stress, followed by both power law indices and amplitude sweep moduli.

For the calcium-varied nanocomposite compositions, the static spreadability showed strong correlations with shear viscosity and calcium concentration, followed by formulation pH, amplitude sweep moduli, and the power law flow index. Dynamic spreadability was found to be strongly correlated with static spreadability, water percentage, calcium concentration, material pH, and power law flow index, which are followed by yield stress, frequency moduli, and complex viscosity.

The static spreadability of citric acid-varied nanocomposite compositions were most strongly correlated with shear viscosity, citric acid concentration, material pH, water percentage, and the power law consistency index. However, the dynamic spreadability was most correlated static spreadability, citric acid concentration, power law consistency index, and water percentage, followed by material pH, complex viscosity, and frequency moduli.

Conceptually, it is well-known in dermal-related industries that spreading rheology and sensorial properties are highly influenced by additive/water composition, yield stress, shear viscosity, thixotropy, pH, and linear viscoelastic moduli [71,72]. Furthermore, each of the additives play roles in the spreading nature of these formulations: TiO₂ is a widely used agent in improving the spreadability of cosmetics [73], Spirulina algae yields both viscosifying [74,75] and lubricating properties [74,76], calcium is used as a viscosity control agent [77] for cosmetics, and citric acid is used as a chelating agent [51] which can play a significant role as a viscosifier [78,79]. In particular, chelating agents improve viscosity when incorporated in combination other additives, like citric acid and anionic polymers [80-82]. which is applicable to these nanocomposite compositions since Spirulina biomass is an anionic compound in aqueous environments [75,83,84].

Spreading capabilities will vary depending on the product design and specific application. Static and dynamic spreadability depict different scenarios, where there must be a balance between pressure-loaded spreadability and shear-induced spreadability. For example, nanocomposite compositions with high static spreadability (being highly liquid-like) can result in difficulty when trying to apply the material to a specific area of the skin, whereas highly viscous nanocomposite compositions with reduced shear-thinning (e.g. low dynamic spreadability) would result in difficulties concerning process manufacturing operations. Thus, achieving and characterizing high tunability in formulation chemistry is crucial for every step of the process: including manufacturing processes, shelf-life stability, geographic location, consumer application, storage requirements, and health/safety regulations.

### pH of Nanocomposite Compostions

As previously discussed, it was observed that the Spirulina biomass is anionic in aqueous environments which acidifies the water-based materials in this study. Based on a previous study [75], it was determined that the Spirulina material had a sort of pH buffering property with alkaline and acidic denaturants, despite its residual acidic yield in water. When increasing the concentration of each additive that was studied, TiO₂ was the only additive that displayed increases in the pH of the nanocomposite compositions, while Spirulina, calcium, and citric acid all displayed reductions in formulation pH. These results for TiO₂ [85], Spirulina [75], and calcium [86], and citric acid [87] agree with other findings that are discussed in literature.

For the TiO₂-varied nanocomposite compositions, the pH data was statistically correlated (Table 4A) with the material's amplitude sweep moduli, while the algae-varied formulation pH was significantly correlated (Table 4B) with static spreadability, water percentage, and Spirulina concentration. The pH of calcium-varying composites was significantly correlated (Table 4C) with calcium concentration, water percentage, and shear viscosity, followed by power law flow index, static spreadability, and yield stress. The citric acid-varying formulation pH values were statistically correlated (Table 4D) with static spreadability, shear viscosity, citric acid concentration, and the power law flow index.

### Oscillatory Rheology

### Amplitude Sweep & Yield Stress, Frequency Sweep & Complex Viscosity

In terms of the viscoelastic response of the storage and loss moduli for both amplitude sweep and frequency sweep, it is shown that all nanocomposite compositions display storage moduli that are greater than the loss moduli, which indicates that all nanocomposite compositions behave more like solid-like rather than liquid-like. Additionally, all nanocomposite compositions exhibit very similar yield strains around 1% strain. However, increasing additive concentration increase yields increases in both the stiffness/rigidity and formulation strength of the nanocomposite composition, which varies depending on the particular additive being increased.

Higher concentrations of citric acid (0.5% or 1%) and calcium (4.5% or 6%) yield make the nanocomposite composition highly rigid and strong, which agrees with observations of the material being similar to a paste-like material. On the other hand, reducing the concentration of citric acid (0% or 0.25%) or calcium (1.5% or 3%) results in materials that are observed to be similar to creams. These stiffening and strengthening effects of citric acid are thought to be due to its chelating [51,80-82] and crosslinking properties [80,88,89] with both metals and anionic organic materials, while the stiffening and strengthening effects of calcium are thought to be due to its ionic bridging [90] or crosslinking properties [91-93]. Given the presence of TiO₂ in these nanocomposite compositions and that it acts as a catalyst for crosslinking citric acid with polysaccharides [94,95], it is likely that a similar effect is occurring with the different peptide [96] or polysaccharide [97,98] components of Spirulina in these nanocomposite compositions.

The formulation strength is evaluated in terms of the stress response and the yield stress that is measured during testing, which is directly indicative of the force needed to squeeze a product from a tube and how it handles before applying to skin or other surfaces. Given the range of stiffness and strength measured between the different formulations, it is understood that these formulations can be manufactured to meet various desirable stiffness and strength demands for different consumer products.

The amplitude sweep moduli of the TiO₂-varied nanocomposite compositions have strong correlations (Table 4A) with TiO₂ concentration, shear viscosity, water percentage, and yield stress. Additionally, the yield stress values were correlated with shear viscosity and both power law indices, followed by amplitude sweep moduli, frequency sweep moduli, and complex viscosity. Frequency sweep moduli and complex viscosity were correlated with both power law indices, followed by shear viscosity and yield stress.

The most significant correlation for amplitude sweep moduli for the algae-varied nanocomposite compositions was shear viscosity, while the yield stress values were correlated with shear viscosity, frequency sweep moduli, and complex viscosity, followed by amplitude sweep moduli and the power law flow index. Furthermore, the frequency sweep moduli and complex viscosity were statistically correlated (Table 4B) with shear viscosity, yield stress, complex viscosity, and both power law indices, followed by the amplitude sweep moduli.

### Formulation pH, Water Percentage, and Spirulina Concentration.

For the calcium-varied formulations, the amplitude sweep moduli showed strong correlations (Table 4C) with frequency sweep moduli, complex viscosity, and yield stress, followed by static spreadability and power law flow index. Yield stress values were found to be strongly correlated with frequency sweep moduli, complex viscosity, and the power law flow index, followed by shear viscosity, calcium concentration, formulation pH, amplitude sweep moduli, and water percentage. Additionally, frequency sweep moduli and complex viscosity were correlated with complex viscosity, power law flow index, and yield stress, followed by amplitude sweep moduli and shear viscosity.

The amplitude sweep moduli of citric acid-varied nanocomposite compositions were most strongly correlated (Table 4D) with water percentage, citric acid concentration, and yield stress. For yield stress values, the strongest correlations were with amplitude sweep moduli. Furthermore, the frequency sweep moduli and complex viscosity were found to be significantly correlated with the power law consistency index.

### Rotational Rheology

### Flow Curves

Shear-thinning behavior was present for all nanocomposite compositions measured, where increasing any additive concentration results in increased viscosity values and more pronounced shear-thinning properties. Majority (˜99%) of the viscosity declination is exhibited within the approximate shear range of 1-1,250 s⁻¹, while the approximate shear range of 1,250-10,000 s⁻¹ shows the remaining viscosity reductions. Although all formulations experienced increases in viscosity as additive concentration, the most pronounced increases in viscosity during high shear rates (250-10,000 s⁻¹) were attributed to the increase in Spirulina algae concentration, while increasing the citric acid concentration yielded the most pronounced increases in viscosity at low shear rates (1-250 s⁻¹). The statistical correlations for shear viscosity values at 2,000 s⁻¹ were discussed previously in section 4.2 and denoted as “dynamic spreadability”.

As previously discussed, the shear viscosity data was plotted and fitted with the power law model to determine the power law flow index “n” and consistency index “k” for the 1,000-10,000 s⁻¹ shear rate regime, which is relevant for both the shear regime of spreading topical products on skin as well as the shear regime that can be experienced during manufacturing processes. Decreasing the flow index “n” between the range of 0<n<1 indicates that the shear-thinning characteristics of the non-Newtonian materials is being enhanced [99]. Compared to the other nanocomposite composition, the citric acid-varying nanocomposite compositions depict the most significant decrease in flow index “n”, which means that increasing citric acid concentration has the most pronounced shear-thinning effect that was investigated in this study. In contrast to this, the calcium-varying nanocomposite compositions show the least reduction in flow index “n” values, which means that these nanocomposite compositions have the least pronounced shear-thinning effect as calcium concentration increases. Given that consistency index “k’ is representative of a material's pumpability [100], the algae-varying nanocomposite compositions depict the most significant increases in its value, while the calcium-varying nanocomposite compositions depict the lowest increases in consistency index value. Thus, the algae and citric acid concentrations have the largest impacts on pumpability, while TiO₂ concentration and calcium concentration has lower impacts on pumpability.

The most significant correlations (Table 4A) for power law flow index with the TiO₂-varied nanocomposite compositions are yield stress and shear viscosity, followed by frequency moduli, complex viscosity, and the consistency index. Similarly, the power law consistency index was most significant with frequency sweep moduli and complex viscosity, followed by yield stress, shear viscosity, and the flow index.

The power law flow index of the algae-varied nanocomposite compositions has strong correlations (Table 4B) with yield stress and shear viscosity, followed by amplitude moduli, frequency moduli, complex viscosity, and consistency index. Additionally, the power law consistency index of these formulations has strong correlations with yield stress and shear viscosity, followed by frequency sweep moduli, complex viscosity, and the flow index.

The power law flow index of calcium-varied nanocomposite compositions are most strongly correlated (Table 4C) with shear viscosity and yield stress, followed by calcium concentration, pH, water percentage, static spreadability, amplitude moduli, frequency moduli, complex viscosity, and the consistency index. However, the power law consistency index is only significantly correlated with the flow index.

For the citric acid-varied nanocomposite compositions, the power law flow index only showed a strong correlation (Table 4D) with the formulation pH. However, the power law consistency index was found to be strongly correlated with shear viscosity and static spreadability, followed by citric acid concentration, water percentage, frequency moduli, and complex viscosity.

### Transient Rheology

### Oscillatory 3-Interval-Thixotropy and Creep—Recovery

Thixotropy and creep—recovery are important parameters for the evaluation of nanocomposite compositions. In particular, a fluid's thixotropic behavior can greatly influence the mixing process, whether it be by making the manufacturing process easier [101] or by damaging machinery (e.g. mixing impellers), which is heavily reliant on the accuracy of the pump and flow designs. Another important parameter for cosmetic formulations is zero-shear viscosity [102], which can be determined through the creep—recovery measurements [103]. Zero-shear viscosity is indicative of a nanocomposite composition's stability [104] and microstructure [105] during storage, which is important considering the fact that cosmetic formulations spend most of their time inside a container at zero-shear conditions.

Regarding the strain thixotropy measurements with loading and recovery intervals at 1% strain, it can be determined that higher water percentage and/or higher algae concentration will lead to a quicker structural recovery at 20° C. conditions. Additionally, it is seen that high concentrations of calcium (6 wt %) and citric acid (1 wt %) showed minimal structure recovery at 20° C. after 15 hours, but significant structural strength recovery when increased to 70° C. after only 2 hours. In particular, the calcium-varying nanocomposite compositions had an increased strength recovery of approximately 2,693.04% within 2 hours, and the citric acid-varying f nanocomposite compositions had an increased strength recovery of an approximate 135.50%. The drastic increase in recovery with the application of heat is beneficial for processing and manufacturing where the formulations can be prepared accordingly and then heated to speed-up the strengthening process, which will significantly improve cycle time and efficiency. For calcium-loaded materials regarding heat-induced material strengthening, similar observations in literature labeled this effect as a thermal switching [106] or thermal stiffening [107] characteristic. On the other hand, citric acid crosslinks with polymeric components (e.g. peptides and polysaccharides), which can be improved at elevated temperatures, in addition to chelating with metals (e.g. calcium) [108] and adsorbing with nanoparticles (e.g. titanium dioxide) [94,95].

Based on the creep—recovery measurements with a constant stress load of 50 Pa, it can be determined that lower water percentage and/or higher additive concentration will result in an increase in structural recovery and increases in zero-shear viscosity. Over the 15-hour recovery interval, the algae-varied nanocomposite compositions had an approximate recovery of 52.05% and the calcium-varied nanocomposite compositions had a recovery of approximately 49.58%, where it is understood that these two formulations were able to recovery more if recovery time was extended past 15 hours. The TACC formulation with 1 wt % citric acid concentration had the highest zero-shear viscosity, followed closely by the TAC formulation with 6 wt % calcium. These results indicate that the drastic increase in material strength is due to citric acid's chelating/crosslinking capabilities [51,80-82,88,89] between the TiO₂ [94,95], constituents of Spirulina, and calcium ions [108]. In absence of the citric acid component, the increase in material strength based on the increase in calcium concentration is likely due to its ionic bridging [90] and/or crosslinking properties [91].

A novel algae-based nanocomposite composition was used for the first time to develop a highly tunable and biocompatible cosmetic dermal product, which has the potential to completely eliminate the need to incorporate chemical surfactants in order to meet rheological or sensorial demands. The rheological and sensorial properties of this formulation were characterized to determine its tunability by testing the changes in their properties at different concentrations of each additive. Each component has one or more beneficial contributions to skin health, including anti-inflammatory properties, anti-bacterial properties, skin tissue regeneration, dermal pigment lightening, natural chemical exfoliants, promoting mineral absorption, improving skin homeostasis, maintenance of acid mantle functionality, and/or serving as a loading carrier for other hydrophilic and/or lipophilic compounds.

The pH measurements indicated that increasing the concentration of each additive will have a distinct effect on the formulation pH. In particular, the formulation pH would decrease as any additive concentration increased, except TiO₂ which displayed that increases in concentration resulted in increases of formulation pH. Based on this knowledge, the pH of these materials can be tuned and refined to meet consumer demands in the market.

The viscoelastic measurements determined that the nanocomposite compositions can behave like viscoelastic solids where the storage modulus exceeds the loss modulus throughout the LVER during amplitude sweep tests and the entire range measured in frequency sweep tests. The formulations containing the highest concentrations of calcium (4.5% and 6%) and citric acid (0.5% and 1%) displayed the highest moduli values which is due to increased ionic bridging, chelation, and/or crosslinking involved within the formulation microstructures.

For shear viscosity measurements, it is confirmed that all f nanocomposite compositions exhibit shear-thinning behavior that can be modeled with the power-law equation between the range between 1,000-10,000 s⁻¹, which incorporates high shear for both dermal application and manufacturing processes, respectively. The magnitude of viscosity will generally increase with the increase of any additive concentration. However, adding Spirulina results in the highest viscosity at high-shear rates (250-10,000 s⁻¹), while adding citric acid results in the highest viscosity at low-shear rates (1-250 s⁻¹).

From the transient rheological measurements, it was found that increasing the water percentage or algae concentration of the composite could improve the structural thixotropic recovery of the nanocomposite compositions at 20° C. Although, by incorporating energy into the system via heat, the structural recovery times were drastically reduced. Increasing calcium or citric acid concentration along with system heat will further reduce the necessary recovery time, which indicates the presence of a thermal switching/stiffening property. Regarding zero-shear viscosity calculations from the creep—recovery tests, all nanocomposite compositions that were tested exhibited zero-shear viscosity values that were indicative of formulation with good stability and microstructure in terms storage capabilities. Additionally, increasing additive concentration resulted in higher zero-shear viscosity values where increasing calcium or citric acid concentration resulted in the most pronounced increases zero-shear viscosity, most likely due to being bridging and/or crosslinking agents.

In terms of which additive concentration is being varied, the static and dynamic spreadability performance of these materials significantly correlated with different parameters. In terms of additive type, TiO₂ nanoparticles are essentially solid spheres and the Spirulina powder used is highly colloidal when dispersed in water, while calcium and citric acid are fully dissolved constituents. In terms of static spreadability, nanocomposite compositions with varying TiO₂ or Spirulina concentration have strong correlations with additive concentration, pH, and water percentage, while their dynamic spreadability have strong correlations with the viscoelastic moduli, yield stress, and power law indices. On the other hand, regarding the static and dynamic spreadability performance of nanocomposite compositions with varying calcium and citric acid concentration, it is concluded that the two performances are strongly correlated with each other, additive concentration, pH, and water percentage. Given that the additives can contribute different correlating effects and effect intensity to the formulation sensory characteristics, it is well understood that this cosmetic formulation has great potential as a tunable, biocompatible, and surfactant-free composite with the sophisticated ability to meet various rheological demands for dermal applications.

One of ordinary skill in the art will readily appreciate that alternative but functionally equivalent components, materials, designs, and equipment can be used. The inclusion of additional elements can be deemed readily apparent and obvious to one of ordinary skill in the art. Specific elements disclosed herein are not to be interpreted as limiting, but rather as a basis for the claims and as a representative basis for teaching one of ordinary skill in the art to employ the present invention. Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present invention is intended to embrace all such alternatives, modifications, and variations as fall within the scope of the claims, together with all equivalents thereof.

As used herein, the singular forms “a”, “an”, and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”. As used herein, use of the term “including” as well as other forms, such as “includes,” and “included,” is not limiting.

Certain embodiments and features have been described using a set of numerical upper limits and a set of numerical lower limits. It should be appreciated that ranges including the combination of any two values, e.g., the combination of any lower value with any upper value, the combination of any two lower values, and/or the combination of any two upper values are contemplated unless otherwise indicated. It should also be appreciated that the numerical limits can be the values from the examples. Certain lower limits, upper limits and ranges appear in at least one claims below. All numerical values are “about” or “approximately” the indicated value, and consider experimental error and variations that would be expected by a person having ordinary skill in the art. Unless otherwise indicated, all numbers expressing feature sizes, amounts, and physical properties used in the specification and claims are to be understood as being modified in all instances by the term “about.” The term “about” is defined to be ±2% of the modified value.

It is understood that any specific order or hierarchy of steps in the processes disclosed is an illustration of example approaches. Based upon design preferences, it is understood that the specific order or hierarchy of steps in the processes can be rearranged, or that all illustrated steps be performed. Some of the steps can be performed simultaneously. For example, in certain circumstances, multitasking and parallel processing can be advantageous. Moreover, the separation of various system components illustrated above should not be understood as requiring such separation, and it should be understood that the described program components and systems can generally be integrated together in a single software product or packaged into multiple software products.

All patents, patent applications, provisional applications, and publications referred to or cited herein are incorporated by reference in their entirety, including all figures and tables, to the extent they are not inconsistent with the explicit teachings of this specification.

### REFERENCES

[1] Benhur A M, Pingali S, Amin S. Application of Biosurfactants and Biopolymers in Sustainable Cosmetic Formulation Design. J Cosmet Sci 2020; 71:455-80.
[2] Hitce J, Xu J, Brossat M, Frantz M-C, Dublanchet A-C, Philippe M, et al. UN sustainable development goals: How can sustainable/green chemistry contribute? Green chemistry as a source of sustainable innovations in the cosmetic industry. Curr Opin Green Sustain Chem 2018; 13:164-9. https://doi.org/https://doi.org/10.1016/j.cogsc.2018.06.019.
[3] Ikkyung S. Interdisciplinary Literaure Analysis between Cosmetic Container Design and Customer Purchasing Intention. [symbol] 2021; 12:21-9. https://doi.org/10.13106/JIDB.2021. VOL12.NO3.21.
[4] Azmi Hassali M, AL-Tamimi S K. Malaysian Cosmetic Market: Current and Future Prospects. Pharm Regul Aff Open Access 2015; 04. https://doi.org/10.4172/2167-7689.1000155.
[5] Cosmetics Market Size to Worth Around US $480.4 Bn by 2030 n.d. https://www.globenewswire.com/news-release/2021/10/05/2308851/0/en/Cosmetics-Market-Size-to-Worth-Around-US-480-4-Bn-by-2030.html (accessed May 27, 2022).
[6] Venkataramani D, Tsulaia A, Amin S. Fundamentals and applications of particle stabilized emulsions in cosmetic formulations. Adv Colloid Interface Sci 2020; 283:102234. https://doi.org/10.1016/j.cis.2020.102234.
[7] Hayase M. Chapter 10—Introduction to Cosmetic Materials. In: Sakamoto K, Lochhead R Y, Maibach H I, Yamashita YBT-C S and T, editors, Amsterdam: Elsevier; 2017, p. 149-54. https://doi.org/https://doi.org/10.1016/B978-0-12-802005-0.00010-0.
[8] Hosoi J, Koyama J, Ozawa T. Chapter 5—New Aspects of Cosmetics and Cosmetic Science. In: Sakamoto K, Lochhead R Y, Maibach H I, Yamashita YBT-C S and T, editors, Amsterdam: Elsevier; 2017, p. 87-100. https://doi.org/https://doi.org/10.1016/B978-0-12-802005-0.00005-7.
[9] Huber P. Chapter 37—Sensory Measurement—Evaluation and Testing of Cosmetic Products. In: Sakamoto K, Lochhead R Y, Maibach H I, Yamashita YBT-C S and T, editors, Amsterdam: Elsevier; 2017, p. 617-33. https://doi.org/https://doi.org/10.1016/B978-0-12-802005-0.00037-9.
[10] Binks B P. Particles as surfactants-similarities and differences. Curr Opin Colloid Interface Sci 2002; 7:21-41. https://doi.org/https://doi.org/10.1016/S1359-0294(02)00008-0.
[11] Naves L, Dhand C, Almeida L, Rajamani L, Ramakrishna S, Soares G. Poly(lactic-co-glycolic) acid drug delivery systems through transdermal pathway: an overview. Prog Biomater 2017; 6:1-11. https://doi.org/10.1007/s40204-017-0063-0.
[12] Pickering S U. CXCVI.-Emulsions. J Chem Soc Trans 1907; 91:2001-21. https://doi.org/10.1039/CT9079102001.
[13] Levine S, Bowen B D, Partridge S J. Stabilization of emulsions by fine particles I. Partitioning of particles between continuous phase and oil/water interface. Colloids and Surfaces 1989; 38:325-43. https://doi.org/https://doi.org/10.1016/0166-6622(89)80271-9.
[14] Varvaresou A, Iakovou K. Biosurfactants in cosmetics and biopharmaceuticals. Lett Appl Microbiol 2015; 61:214-23. https://doi.org/https://doi.org/10.1111/lam.12440.
[15] Yao M, Patel J. Rheological Characterization of Body Lotions. Appl Rheol 2001; 11:83-8. https://doi.org/10.1515/arh-2001-0005.
[16] Morivkovi T, Stern P. Rheological and Textural Properties of Cosmetic Emulsions. Appl Rheol 2011; 21. https://doi.org/10.3933/ApplRheol-21-35200.
[17] Sikora M, Juszczak L, Sady M, Krawontka J. Use of starch/xanthan gum combinations as thickeners of cocoa syrups. Nahrung 2003; 47:106-13. https://doi.org/10.1002/food.200390019.
[18] Rao M A. Measurement of Flow and Viscoelastic Properties BT—Rheology of Fluid and Semisolid Foods: Principles and Applications. In: Rao M A, editor, Boston, MA: Springer US; 2007, p. 59-151. https://doi.org/10.1007/978-0-387-70930-7_3.
[19] Brummer R, Godersky S. Rheological studies to objectify sensations occurring when cosmetic emulsions are applied to the skin. Colloids Surfaces A Physicochem Eng Asp 1999; 152:89-94. https://doi.org/https://doi.org/10.1016/S0927-7757(98)00626-8.
[20] Kwak M-S, Ahn H-J, Song K-W. Rheological investigation of body cream and body lotion in actual application conditions. Korea-Australia Rheol J 2015; 27:241-51. https://doi.org/10.1007/si3367-015-0024-x.
[21] Chirdon W M. Utilization of biorefinery waste proteins as feed, glues, composites, and other co-products. Algal Biorefineries Vol. 2 Prod. Refin. Des., 2015. https://doi.org/10.1007/978-3-319-20200-6_11.
[22] Almomani F, Judd S, Bhosale R R, Shurair M, Aljaml K, Khraisheh M. Intergraded wastewater treatment and carbon bio-fixation from flue gases using Spirulina platensis and mixed algal culture. Process Saf Environ Prot 2019; 124:240-50. https://doi.org/https://doi.org/10.1016/j.psep.2019.02.009.
[23] de Marco Castro E, Shannon E, Abu-Ghannam N. Effect of Fermentation on Enhancing the Nutraceutical Properties of Arthrospira platensis (Spirulina). Ferment 2019; 5. https://doi.org/10.3390/fermentation5010028.
[24] Spirulina Market to Grow at a CAGR of 13.6% to Reach n.d. https://www.globenewswire.com/news-release/2020/02/10/1982331/0/en/Spirulina-Market-to-Grow-at-a-CAGR-of-13-6-to-Reach-68-025-2-tons-by-2025-with-Expanding-Application-Areas-of-Phycocyanin.html (accessed Apr. 25, 2022).
[25] Farooq S M, Boppana N B, Devarajan A, Sekaran S D, Shankar E M, Li C, et al. C-phycocyanin confers protection against oxalate-mediated oxidative stress and mitochondrial dysfunctions in MDCK cells. PLoS One 2014; 9:e93056. https://doi.org/10.1371/journal.pone.0093056.
[26] Neyrinck A M, Taminiau B, Walgrave H, Daube G, Cani P D, Bindels L B, et al. Spirulina Protects against Hepatic Inflammation in Aging: An Effect Related to the Modulation of the Gut Microbiota? Nutrients 2017; 9:633. https://doi.org/10.3390/nu9060633.
[27] Liu P, Lee M-K, Choi J-W, Choi Y-H, Nam T-J. Crude protein from Spirulina increases the viability of CCD-986sk cells via the EGFR/MAPK signaling pathway. Int J Mol Med 2019; 43:771-8. https://doi.org/10.3892/ijmm.2018.4025.
[28] Barka A, Amira A B, Francis F, Blecker C. Physicochemical characterization of colored soluble protein fractions extracted from Spirulina (Spirulina platensis). Food Sci Technol Int 2018; 24:651-63. https://doi.org/10.1177/1082013218786862.
[29] Esquivel-Hernández D A, López V H, Rodríguez-Rodríguez J, Alemán-Nava G S, Cuéllar-Bermndez S P, Rostro-Alanis M, et al. Supercritical carbon dioxide and microwave-assisted extraction of functional lipophilic compounds from Arthrospira platensis. Int J Mol Sci 2016; 17. https://doi.org/10.3390/ijms17050658.
[30] Misbahuddin M, Islam AZMM, Khandker S, Ifthaker-Al-Mahmud, Islam N, Anjumanara. Efficacy of Spirulina extract plus zinc in patients of chronic arsenic poisoning: a randomized placebo-controlled study. Clin Toxicol (Phila) 2006; 44:135-41. https://doi.org/10.1080/15563650500514400.
[31] Marangoni A, Foschi C, Micucci M, Nahui Palomino R A, Gallina Toschi T, Vitali B, et al. In vitro activity of Spirulina platensis water extract against different Candida species isolated from vulvo-vaginal candidiasis cases. PLoS One 2017; 12:e0188567-e0188567. https://doi.org/10.1371/journal.pone.0188567.
[32] Józsa L, Ujhelyi Z, Vasvári G, Sinka D, Nemes D, Fenyvesi F, et al. Formulation of Creams Containing Spirulina Platensis Powder with Different Nonionic Surfactants for the Treatment of Acne Vulgaris. Mol 2020; 25. https://doi.org/10.3390/molecules25204856.
[33] Bohlin, L., Rolfsen, W., & Vasange M. Use of sulphoquinovosyldiacylglycerols for the treatment of inflammatory skin diseases. European patent specifics: EP0952836B1, 2004.
[34] Nurmukhambetov Z, Ibrayeva T, Nurmukhambetov A, Bazarbekov Y. Optimization of the Treatment of Chronic Eczema in the Elderly. Int J Pharma Med Biol Sci 2020; 9:91-5. https://doi.org/10.18178/ijpmbs.9.2.91-95.
[35] Völz H G, Kischkewitz J, Woditsch P, Westerhaus A, Griebler W-D, De Liedekerke M, et al. Pigments, Inorganic. Ullmann's Encycl Ind Chem 2006. https://doi.org/https://doi.org/10.1002/14356007.a20_243.pub2.
[36] Raeisi M, Kazerouni Y, Mohammadi A, Hashemi M, Hejazi I, Seyfi J, et al. Superhydrophobic cotton fabrics coated by chitosan and titanium dioxide nanoparticles with enhanced antibacterial and UV-protecting properties. Int J Biol Macromol 2021; 171:158-65. https://doi.org/https://doi.org/10.1016/j.ijbiomac.2020.12.220.
[37] Gad M M, Abualsaud R. Behavior of PMMA Denture Base Materials Containing Titanium Dioxide Nanoparticles: A Literature Review. Int J Biomater 2019; 2019:6190610. https://doi.org/10.1155/2019/6190610.
[38] Yusuf M A, Mahmoud N D, Rirash F R, Stoff B K, Liu Y, McMichael J R. Skin lightening practices, beliefs, and self-reported adverse effects among female health science students in Borama, Somaliland: A cross-sectional survey. Int J Women's Dermatology 2019; 5:349-55. https://doi.org/https://doi.org/10.1016/j.ijwd.2019.08.006.
[39] Rai M, Ingle A P, Gupta I, Pandit R, Paralikar P, Gade A, et al. Smart nanopackaging for the enhancement of food shelf life. Environ Chem Lett 2019; 17:277-90. https://doi.org/10.1007/s10311-018-0794-8.
[40] Color Additives Permitted for Use in Cosmetics|FDA n.d. https://www.fda.gov/cosmetics/cosmetic-ingredient-names/color-additives-permitted-use-cosmetics (accessed Apr. 7, 2022).
[41] Denda M, Fuziwara S, Inoue K. Influx of calcium and chloride ions into epidermal keratinocytes regulates exocytosis of epidermal lamellar bodies and skin permeability barrier homeostasis. J Invest Dermatol 2003; 121:362-7. https://doi.org/10.1046/j.1523-1747.2003.12367.x.
[42] Morrison S J, Spradling A C. Stem cells and niches: mechanisms that promote stem cell maintenance throughout life. Cell 2008; 132:598-611. https://doi.org/10.1016/j.cell.2008.01.038.
[43] Rinnerthaler M, Streubel M K, Bischof J, Richter K. Skin aging, gene expression and calcium. Exp Gerontol 2015; 68:59-65. https://doi.org/10.1016/j.exger.2014.09.015.
[44] Morris C A, Theis R L, Miller R K, Acuff G R, Savell J W. Improving the flavor of calcium chloride and lactic acid injected mature beef top round steaks. Meat Sci 1997; 45:531-7. https://doi.org/https://doi.org/10.1016/S0309-1740(96)00110-6.
[45] CFR—Code of Federal Regulations Title 21 n.d. https://www.accessdata.fda.gov/scripts/cdrh/cfdocs/cfcfr/CFRSearch.cfm?fr=184.1193&SearchT erm=calcium chloride (accessed Apr. 7, 2022).
[46] CFR—Code of Federal Regulations Title 21 n.d. https://www.accessdata.fda.gov/scripts/cdrh/cfdocs/cfcfr/CFRSearch.cfm?fr=178.1010&SearchT erm=calcium chloride (accessed Apr. 7, 2022).
[47] de Mendonga A J G, Vaz M I P M, de Mendonga D I M D. Activity coefficients in the evaluation of food preservatives. Innov Food Sci Emerg Technol 2001; 2:175-9. https://doi.org/https://doi.org/10.1016/S1466-8564(01)00037-6.
[48] Safety assessment of Salicylic Acid, Butyloctyl Salicylate, Calcium Salicylate, C12-15 Alkyl Salicylate, Capryloyl Salicylic Acid, Hexyldodecyl Salicylate, Isocetyl Salicylate, Isodecyl Salicylate, Magnesium Salicylate, MEA-Salicylate, Ethylhexyl Salicyl. Int J Toxicol 2003; 22 Suppl 3:1-108.
[49] Mishra S K, Shrivastav A, Mishra S. Effect of preservatives for food grade C-PC from Spirulina platensis. Process Biochem 2008; 43:339-45. https://doi.org/https://doi.org/10.1016/j.procbio.2007.12.012.
[50] FISCHER J R, FLETCHER D L, COX N A, BAILEY J S. Microbiological Properties of Hard-Cooked Eggs in a Citric Acid-Based Preservative Solution. J Food Prot 1985; 48:252-6. https://doi.org/10.4315/0362-028X-48.3.252.
[51] Brul S, Coote P. Preservative agents in foods: Mode of action and microbial resistance mechanisms. Int J Food Microbiol 1999; 50:1-17. https://doi.org/https://doi.org/10.1016/S0168-1605(99)00072-0.
[52] Ionidis G, Hubscher J, Jack T, Becker B, Bischoff B, Todt D, et al. Development and virucidal activity of a novel alcohol-based hand disinfectant supplemented with urea and citric acid. BMC Infect Dis 2016; 16:77. https://doi.org/10.1186/s12879-016-1410-9.
[53] Krug P W, Larson C R, Eslami A C, Rodriguez L L. Disinfection of foot-and-mouth disease and African swine fever viruses with citric acid and sodium hypochlorite on birch wood carriers. Vet Microbiol 2012; 156:96-101. https://doi.org/https://doi.org/10.1016/j.vetmic.2011.10.032.
[54] Ciriminna R, Meneguzzo F, Delisi R, Pagliaro M. Citric acid: emerging applications of key biotechnology industrial product. Chem Cent J 2017; 11:22. https://doi.org/10.1186/s13065-017-0251-y.
[55] Saxena V, Yadav K. Glycolic Acid, Lactic Acid, Mandelic Acid, Salicylic Acid, Citric Acid, Gluconolactone: Skin Exfoliators in Combination Therapy of Acne Vulgaris. Int J Res Eng Sci Manag 2020; 3:54-5. https://doi.org/10.47607/ijresm.2020.334.
[56] Kapuscinska A, Nowak I. [Use of organic acids in acne and skin discolorations therapy]. Postepy Hig Med Dosw (Online) 2015; 69:374-83. https://doi.org/10.5604/17322693.1145825.
[57] Packianathan N, Kandasamy R. Skin Care with Herbal Exfoliants. Funct Plant Sci Biotechnol 2011; 5:94-7.
[58] Lönnerdal B. Dietary Factors Influencing Zinc Absorption. J Nutr 2000; 130:13785-13835. https://doi.org/10.1093/jn/130.5.1378S.
[59] Kasouni A I, Chatzimitakos T G, Troganis A N, Stalikas C D. Citric acid-based carbon dots: From revealing new insights into their biological properties to demonstrating their enhanced wound healing potential by in vitro and in vivo experiments. Mater Today Commun 2021; 26:102019. https://doi.org/https://doi.org/10.1016/j.mtcomm.2021.102019.
[60] Davis J L, Jeansonne B G, Davenport W D, Gardiner D. The Effect of Irrigation with Doxycycline or Citric Acid on Leakage and Osseous Wound Healing. J Endod 2003; 29:31-5. https://doi.org/https://doi.org/10.1097/00004770-200301000-00009.
[61] Xie Z, Aphale N V, Kadapure T D, Wadajkar A S, Orr S, Gyawali D, et al. Design of antimicrobial peptides conjugated biodegradable citric acid derived hydrogels for wound healing. J Biomed Mater Res Part A 2015; 103:3907-18. https://doi.org/https://doi.org/10.1002/jbm.a.35512.
[62] Wu X, Dai H, Xu C, Liu L, Li S. Citric acid modification of a polymer exhibits antioxidant and anti-inflammatory properties in stem cells and tissues. J Biomed Mater Res Part A 2019; 107:2414-24. https://doi.org/https://doi.org/10.1002/jbm.a.36748.
[63] Surapaneni K M, Jainu M. Comparative effect of pioglitazone, quercetin and hydroxy citric acid on the status of lipid peroxidation and antioxidants in experimental non-alcoholic steatohepatitis. J Physiol Pharmacol 2014; 65:67-74.
[64] Murakami A, Nakamura Y, Tanaka T, Kawabata K, Takahashi D, Koshimizu K, et al. Suppression by citrus auraptene of phorbol ester- and endotoxin-induced inflammatory responses: role of attenuation of leukocyte activation. Carcinogenesis 2000; 21:1843-50. https://doi.org/10.1093/carcin/21.10.1843.
[65] Bowes L. The science of hydroxy acids: mechanisms of action, types and cosmetic applications. J Aesthetic Nurs 2013; 2:77-81. https://doi.org/10.12968/joan.2013.2.2.77.
[66] CFR—Code of Federal Regulations Title 21 n.d. https://www.accessdata.fda.gov/scripts/cdrh/cfdocs/cfcfr/CFRSearch.cfm?fr=184.1033 (accessed Apr. 16, 2022).
[67] CFR—Code of Federal Regulations Title 21 n.d. https://www.accessdata.fda.gov/scripts/cdrh/cfdocs/cfcfr/CFRSearch.cfm?fr=172.832 (accessed Apr. 16, 2022).
[68] Microorganisms & Microbial-Derived Ingredients Used in Food (Partial List)|FDA n.d. https://www.fda.gov/food/generally-recognized-safe-gras/microorganisms-microbial-derived-ingredients-used-food-partial-list (accessed Apr. 16, 2022).
[69] Federal Register::Guidance for Industry: Labeling for Topically Applied Cosmetic Products Containing Alpha Hydroxy Acids as Ingredients; Availability n.d. https://www.federalregister.gov/documents/2005/01/10/05-381/guidance-for-industry-labeling-for-topically-applied-cosmetic-products-containing-alpha-hydroxy (accessed Apr. 16, 2022).
[70] CFR—Code of Federal Regulations Title 21 n.d. https://www.accessdata.fda.gov/scripts/cdrh/cfdocs/cfcfr/CFRSearch.cfm?fr=582.6033 (accessed Apr. 16, 2022).
[71] US Food and Drug Administration. Draft Guidance on Acyclovir Cream. Fda 2016; 1:1-6.
[72] European Medicines Agency. Draft Guideline on quality and equivalence of topical products. Ema/Chmp/Qwp/708282/2018 2018; 44:1-36.
[73] Singh P, Nanda A. Enhanced sun protection of nano-sized metal oxide particles over conventional metal oxide particles: an in vitro comparative study. Int J Cosmet Sci 2014; 36:273-83. https://doi.org/10.1111/ics.12124.
[74] Thibodeaux G M. Application of a Proteinaceous Algal Waste as a Drilling Fluid Additive. University of Louisiana at Lafayette, 2019.
[75] Schexnayder P, Baudoin N, Chirdon W M. Enhanced oil recovery from denatured algal biomass: Synergy between conventional and emergent fuels. Fuel 2020:119533. https://doi.org/10.1016/j.fuel.2020.119533.
[76] D'Addona D M, Conte S, Teti R, Marzocchella A, Raganati F. Feasibility study of using microorganisms as lubricant component in cutting fluids. Procedia CIRP 2020; 88:606-11. https://doi.org/https://doi.org/10.1016/j.procir.2020.05.106.
[77] Organisation for Economic Co-Operation and Development Screening Information Data Sets (OECD SIDS). Calcium Chloride CAS No.: 10043-52-4 2002:1-154.
[78] Elkatatny S. Evaluating the Chemical Reaction of Chelating Agents with Xanthan Gum. Arab J Sci Eng 2017; 42:1427-34. https://doi.org/10.1007/s13369-016-2299-8.
[79] Kamal M shahzad, Mohammed M, Elkatatny S. Development of Chelating Agent-Based Polymeric Gel System for Hydraulic Fracturing. Energies 2018; 11:1663. https://doi.org/10.3390/enl 1071663.
[80] Simões B M, Cagnin C, Yamashita F, Olivato J B, Garcia P S, de Oliveira S M, et al. Citric acid as crosslinking agent in starch/xanthan gum hydrogels produced by extrusion and thermopressing. LWT 2020; 125:108950. https://doi.org/https://doi.org/10.1016/j.lwt.2019.108950.
[81] Petitjean M, Aussant F, Vergara A, Isasi J R. Solventless crosslinking of chitosan, xanthan, and locust bean gum networks functionalized with β-cyclodextrin. Gels 2020; 6:1-13. https://doi.org/10.3390/gels6040051.
[82] Bueno V B, Bentini R, Catalani L H, Petri D F S. Synthesis and swelling behavior of xanthan-based hydrogels. Carbohydr Polym 2013; 92:1091-9. https://doi.org/https://doi.org/10.1016/j.carbpol.2012.10.062.
[83] Dmytryk A, Saeid A, Chojnacka K. Biosorption of Microelements by Spirulina: Towards Technology of Mineral Feed Supplements. Sci World J 2014; 2014. https://doi.org/10.1155/2014/356328.
[84] Michalak I, Mironiuk M, Godlewska K, Trynda J, Marycz K. Arthrospira (Spirulina) platensis: An effective biosorbent for nutrients. Process Biochem 2020; 88:129-37. https://doi.org/https://doi.org/10.1016/j.procbio.2019.10.004.
[85] Chadwick M D, Goodwin J W, Lawson E J, Mills P D A, Vincent B. Surface charge properties of colloidal titanium dioxide in ethylene glycol and water. Colloids Surfaces A Physicochem Eng Asp 2002; 203:229-36. https://doi.org/https://doi.org/10.1016/S0927-7757(01)01101-3.
[86] Wang S, McDonnell E H, Sedor F A, Toffaletti J G. pH effects on measurements of ionized calcium and ionized magnesium in blood. Arch Pathol Lab Med 2002; 126:947-50. https://doi.org/10.5858/2002-126-0947-PEOMOI.
[87] Haznedaroǧlu F. Efficacy of various concentrations of citric acid at different pH values for smear layer removal. Oral Surgery, Oral Med Oral Pathol Oral Radiol Endodontology 2003; 96:340-4. https://doi.org/https://doi.org/10.1016/S1079-2104(03)00164-1.
[88] Nourbakhsh H, Emam-Djomeh Z, Madadlou A, Mousavi M E, Moosavi-Movahedi A A, Gunasekaran S. Antioxidant Peptidic Particles for Delivery of Gallic Acid. J Food Process Preserv 2017; 41:e12767. https://doi.org/https://doi.org/10.1111/jfpp.12767.
[89] Ghorpade V S, Yadav A V, Dias R J, Mali K K, Pargaonkar S S, Shinde P V, et al. Citric acid crosslinked carboxymethylcellulose-poly(ethylene glycol) hydrogel films for delivery of poorly soluble drugs. Int J Biol Macromol 2018; 118:783-91. https://doi.org/10.1016/j.ijbiomac.2018.06.142.
[90] Carter-Fenk K A, Dommer A C, Fiamingo M E, Kim J, Amaro R E, Allen H C. Calcium bridging drives polysaccharide co-adsorption to a proxy sea surface microlayer. Phys Chem Chem Phys 2021; 23:16401-16. https://doi.org/10.1039/D1CP01407B.
[91] Li Z, Lin Z. Recent advances in polysaccharide-based hydrogels for synthesis and applications. Aggregate 2021; 2:e21. https://doi.org/https://doi.org/10.1002/agt2.21.
[92] Mikula K, Skrzypczak D, Ligas B, Witek-Krowiak A. Preparation of hydrogel composites using Ca2+ and Cu2+ ions as crosslinking agents. SN Appl Sci 2019; 1:643. https://doi.org/10.1007/s42452-019-0657-3.
[93] Lin J, Cai X, Tang M, Wang S. Preparation and Evaluation of the Chelating Nanocomposite Fabricated with Marine Algae Schizochytrium sp. Protein Hydrolysate and Calcium. J Agric Food Chem 2015; 63:9704-14. https://doi.org/10.1021/acs.jafc.5b04001.
[94] Abou-okeil A. Citric Acid Crosslinking of Cellulose Using TiO2 Catalyst by Pad-Dry-Cure Method. Polym Technol Eng—POLYM-PLAST TECHNOL ENG 2008; 47:174-9. https://doi.org/10.1080/03602550701816126.
[95] Mudunkotuwa I A, Grassian V H. Citric Acid Adsorption on TiO2 Nanoparticles in Aqueous Suspensions at Acidic and Circumneutral pH: Surface Coverage, Surface Speciation, and Its Impact on Nanoparticle-Nanoparticle Interactions. J Am Chem Soc 2010; 132:14986-94. https://doi.org/10.1021/ja106091q.
[96] Hongsthong A, Sirijuntarut M, Prommeenate P, Thammathorn S, Bunnag B, Cheevadhanarak S, et al. Revealing differentially expressed proteins in two morphological forms of Spirulina platensis by proteomic analysis. Mol Biotechnol 2007; 36:123-30. https://doi.org/10.1007/s12033-007-0013-5.
[97] Wang B, Cai T, Liu Q, Whitney J C C, Du M, Ma Q, et al. Preparation and evaluation of Spirulina polysaccharide nanoemulsions. Int J Mol Med 2018; 42:1273-82. https://doi.org/10.3892/ijmm.2018.3717.
[98] Li T T, Huang Z R, Jia R B, Lv X C, Zhao C, Liu B. Spirulina platensis polysaccharides attenuate lipid and carbohydrate metabolism disorder in high-sucrose and high-fat diet-fed rats in association with intestinal microbiota. Food Res Int 2021; 147:110530. https://doi.org/10.1016/j.foodres.2021.110530.
[99] Pang B, Wang S, Chen W, Hassan M, Lu H. Effects of flow behavior index and consistency coefficient on hydrodynamics of power-law fluids and particles in fluidized beds. Powder Technol 2020; 366:249-60. https://doi.org/https://doi.org/10.1016/j.powtec.2020.01.061.
[100] Ariaratnam S T, Harbin B C, Stauber R L. Modeling of annular fluid pressures in horizontal boring. Tunn Undergr Sp Technol 2007; 22:610-9. https://doi.org/https://doi.org/10.1016/j.tust.2007.01.001.
[101] Jung S Y, Park J E, Kang T G, Park J D. Flow and mixing analysis of a thixotropic fluid in a barrier-embedded partitioned pipe mixer (BPPM): A numerical study. Int J Heat Mass Transf 2022; 184:122310. https://doi.org/https://doi.org/10.1016/j.ijheatmasstransfer.2021.122310.
[102] Tafuro G, Costantini A, Baratto G, Francescato S, Busata L, Semenzato A. Characterization of polysaccharidic associations for cosmetic use: Rheology and texture analysis. Cosmetics 2021; 8. https://doi.org/10.3390/cosmetics8030062.
[103] Teh J, Lin W, Popuri P. DETERMINATION OF THE ZERO SHEAR VISCOSITY OF POLYETHYLENE. 2015.
[104] Ibanescu C, Danu M, Nanu A, Lungu M, Simionescu B C. Stability of disperse systems estimated using rheological oscillatory shear tests. Rev Roum Chim 2010; 55:933-40.
[105] Dabbaghi M, Namjoshi S, Panchal B, Grice J E, Prakash S, Roberts M S, et al. Viscoelastic and Deformation Characteristics of Structurally Different Commercial Topical Systems. Pharmaceutics 2021; 13. https://doi.org/10.3390/pharmaceutics13091351.
[106] Nonoyama T, Lee Y W, Ota K, Fujioka K, Hong W, Gong J P. Instant Thermal Switching from Soft Hydrogel to Rigid Plastics Inspired by Thermophile Proteins. Adv Mater 2020; 32:1905878. https://doi.org/https://doi.org/10.1002/adma.201905878.
[107] Pacchioni G. Toughened up. Nat Rev Mater 2020; 5:2. https://doi.org/10.1038/s41578-019-0168-2.
[108] Zeiler N, Martinez da Silva L, Romain E, Schäfer D, Jungbunzlauer. Effective Chelation With Citrates and Gluconates 2020; 15:31-3.

What is claimed is:

1. A nanocomposite composition, wherein the composition comprises:
a microalgae or cyanobacterial biomass, wherein the microalgae or cyanobacterial biomass is present in the composition, and wherein the composition has a content of the microalgae or cyanobacterial biomass from about 0.01 wt % to about 95.00 wt %;
nanoparticles, wherein the nanoparticles are present in the composition, wherein the nanoparticles comprise titanium dioxide, and wherein the composition has a content of the nanoparticles from about 0.01 wt % to about 22.84 wt %;
a salt or chelating agent, wherein the salt or chelating agent is present in the composition, and wherein the composition has a content of the nanoparticles from about 0.01 wt % to about 25.01 wt %;
an acid, wherein the acid is present in the composition, and wherein the composition has a content of the acid from about 0.01 wt % to about 2.00 wt %; and
a carrier medium, wherein the carrier medium is present in the composition, and wherein the composition has a content of the carrier medium from about 47.00 wt % to about 68.00 wt %.

2. The nanocomposite composition of claim 1, wherein the microalgae comprises a Spirulina biomass.

3. The nanocomposite composition of claim 1, wherein the salt comprises calcium chloride.

4. The nanocomposite composition of claim 1, wherein the acid comprises an alpha hydroxy acid.

5. The nanocomposite composition of claim 1, wherein the carrier medium comprises water.

6. The nanocomposite composition of claim 1, wherein the nanoparticles have an average diameter from about 1 nm to about 500 nm.

7. The nanocomposite composition of claim 1, wherein the nanocomposite composition has a viscosity from 0.00561 Pa·s to about 1,770 Pa·s within the shear rate range of about 0.1 l/s to about 10,000 l/s.

8. The nanocomposite composition of claim 1, wherein the nanocomposite composition has a yield strength from about 0.18 Pa to about 2,380.00 Pa.

9. A method of making a nanocomposite composition, wherein the method comprises:
contacting a microalgae biomass, nanoparticles, salt or chelating agent, an acid, and a carrier material to make the nanocomposite composition, wherein the composition has a content of the microalgae biomass from about 0.01 wt % to about 95.00 wt %, wherein the nanoparticles comprise titanium dioxide, wherein the composition has a content of the nanoparticles from about 0.01 wt % to about 22.84 wt %, wherein the composition has a content of the salt or chelating agent is from about 0.01 wt % to about 25.01 wt %, wherein the composition has a content of the acid from about 0.01 wt % to about 2.00 wt %, and wherein the composition has a content of the carrier medium from about 47.00 wt % to about 68.00 wt %.

10. The nanocomposite composition of claim 9, wherein the microalgae comprise a Spirulina biomass.

11. The nanocomposite composition of claim 9, wherein the salt comprises calcium chloride.

12. The nanocomposite composition of claim 9, wherein the acid comprises an alpha hydroxy acid.

13. The nanocomposite composition of claim 9, wherein the carrier medium comprises water.

14. The nanocomposite composition of claim 9, wherein the nanoparticles have an average diameter from about 1 nm to about 500 nm.

15. The nanocomposite composition of claim 9, wherein the nanocomposite composition has a viscosity from about 0.1 cP to about 2,500,000.0 cP.

16. The nanocomposite composition of claim 9, wherein the nanocomposite composition has a yield strength from about 0.18 Pa to about 2,380.00 Pa.

17. A method of using a nanocomposite composition, wherein the method comprises:
making a nanocomposite composition, wherein the nanocomposite composition is made by contacting a microalgae biomass, nanoparticles, salt or chelating agent, an acid, and a carrier material to make the nanocomposite composition; wherein the microalgae biomass is present in the composition, wherein the composition has a content of the microalgae or cyanobacterial biomass from about 0.01 wt % to about 95.00 wt %; nanoparticles, wherein the nanoparticles comprise titanium dioxide, wherein the nanoparticles are present in the composition; wherein the composition has a content of the nanoparticles from about 0.01 wt % to about 22.84 wt %; a salt or chelating agent, wherein the salt or chelating agent is present in the composition; wherein the composition has a content of salt or chelating agent from about 0.01 wt % to about 25.01 wt %; an acid, wherein the acid is present in the composition; wherein the composition has a content of the acid from about 0.01 wt % to about 2.0 wt %; and a carrier medium, wherein the carrier medium is present in the composition; and wherein the composition has a content of the carrier medium from about 47.0 wt % to about 68.0 wt %; and
applying to a surface.

18. The method of using a nanocomposite composition of claim 17, wherein the surface is human skin or hair

19. The method of using a nanocomposite composition of claim 17, wherein the microalgae comprise Spirulina biomass.

20. The method of using a nanocomposite composition of claim 17, wherein the nanocomposite composition has a yield strength from about 0.18 Pa to about 2,380.00 Pa.
